(19) United States
(12) Patent Application Publication
(10) Pub. No.: US 2024/0181057 A1
(43) Pub. Date: Jun. 6, 2024

(54) SYNTHETIC INTERMEMBRANE PROTEOLYSIS RECEPTORS FOR CUSTOM ANTIGEN-INDUCED TRANSCRIPTIONAL REGULATION

(71) Applicant: The Regents of the University of California, Oakland, CA (US)

(72) Inventors: Kole T. ROYBAL, San Francisco, CA (US); Iowis ZHU, San Francisco, CA (US); Raymond LIU, San Francisco, CA (US)

(21) Appl. No.: 18/552,141

(22) PCT Filed: Mar. 22, 2022
(86) PCT No.: PCT/US2022/021352

§ 371 Date: Sep. 22, 2023

Related U.S. Application Data
(60) Provisional application No. 63/164,995, filed on Mar. 23, 2021.

(51) Int. Cl. A61K 39/00; A61K 38/00; A61K 45/06; A61K 48/00; C07K 14/47; C07K 14/485; C07K 14/705; C07K 14/715; C07K 16/28; C07K 16/30; C07K 16/32; C07K 16/40; C12N 5/0783; C12N 9/16

(52) U.S. Cl. CPC A61K 39/464482; A61K 39/4611; A61K 39/4612; A61K 39/4633; A61K 39/464404; A61K 39/464406; A61K 39/464412; A61K 39/464466; A61K 39/46447; A61K 45/06; A61K 48/0058; C07K 14/47; C07K 14/4707; C07K 14/485; C07K 14/705; C07K 14/70503; C07K 14/70517; C07K 14/7153; C07K 16/2803; C07K 16/2863; C07K 16/2878; C07K 16/2887; C07K 16/3007; C07K 16/3092; C07K 16/32; C07K 16/40; C12N 5/0636; C12N 9/16; C12Y 301/03048; A61K 38/00; A61K 39/00; C07K 2317/24; C07K 2317/622; C07K 2319/02; C07K 2319/03; C07K 2319/09; C07K 2319/30; C07K 2319/50; C07K 2319/71; C12N 2510/00

(57) ABSTRACT

The present disclosure generally relates to, among other things, a new class of receptors engineered to modulate transcriptional regulation in a ligand-dependent manner. In particular, the new receptors contain a heterologous stop-transfer-sequence and a γ-secretase cleavable transmembrane domain. The disclosure also provides compositions and methods useful for producing such receptors, nucleic acids encoding same, host cells genetically modified with the nucleic acids, as well as methods for modulating an activity of a cell and/or for the treatment of various diseases such as cancers.

### CROSS REFERENCE TO RELATED APPLICATION

The present application claims priority to U.S. Provisional Patent Application Ser. No. 63/164,995 filed on Mar. 23, 2021, the disclosure of which is incorporated by reference herein in its entirety, including any drawings.

### STATEMENT REGARDING FEDERALLY SPONSORED R&D

This invention was made with government support under grant no. OD025751 awarded by The National Institutes of Health. The government has certain rights in the invention.

### INCORPORATION OF THE SEQUENCE LISTING

This application contains a Sequence Listing, which is hereby incorporated herein by reference in its entirety. The accompanying Sequence Listing text file, named “048536-696001WO_Sequence Listing_ST25.txt” was created on Mar. 18, 2022 and is 307 KB.

### FIELD

The present disclosure relates generally to new synthetic cellular receptors that bind cell-surface ligands and have selectable specificities and activities. The disclosure also provides compositions and methods useful for producing such receptors, nucleic acids encoding same, host cells genetically modified with the nucleic acids, as well as methods for modulating an activity of a cell and/or for the treatment of various diseases such as cancers.

### BACKGROUND

An important problem which limits the development of engineered cell therapies in humans is the ability to regulate therapeutic gene expression and engineered cell activity. For example, the first generation of chimeric antigen receptor T cells (CAR-T) lack the ability to modulate or turn off CAR-T activity when needed; other problems include off-target activity and off-tumor/on-target activity (i.e., wherein the CAR-T target antigen is also found on normal cells outside the tumor). One possible solution to these problems is to use a synthetic receptor that is capable of modifying gene expression and/or cellular behavior. Existing synthetic Notch (SynNotch) receptors are capable of binding user-defined cell surface displayed ligands, which triggers proteolytic cleavage of the receptor and release of a transcriptional regulator that induces a custom transcriptional program in the cell. In addition, some existing SynNotch receptors do not require the Notch regulatory regions (NRR) thought to necessary for the induced cleavage of the receptor.

The present disclosure provides, among other things, novel chimeric polypeptides, including a new class of synthetic receptors that do not require sequence from Notch receptors.

### SUMMARY

The present disclosure describes synthetic chimeric polypeptides (e.g., chimeric receptors) that do not require sequence from Notch receptors. In particular, systematic engineering efforts have illustrated that one can utilize (1) a ADAM protease cleavable extracellular domain (e.g., hinge domain from CD8), (2) a γ-secretase cleavable transmembrane domain (TMD), and (3) a stop-transfer-sequence to rationally build synthetic regulated intermembrane proteolysis receptors (SynRIPRs). As described in greater detail below, these receptors can be used to target various therapeutically relevant cell types to sites of disease and regulate the therapeutic activity the cell in a controlled and localized fashion. These receptors can be linked to inducible transcriptional programs such that when the cell senses a disease site it will locally produce therapeutics such as antibodies, cytokines, toxins, and adjuvants. They are a highly versatile and programmable platform for cellular engineering, and essentially the production of any genetically encodable therapeutic can regulated by these receptors.

Provided in some embodiments of the disclosure are synthetic chimeric receptors that exhibit a range of signal characteristics mediated by inclusion of heterologous STS and TMD sequences. These receptors provide a range of sensitivity, including a receptor that is sensitive to the degree of T-cell activation when it is expressed in an activated T cell. Some embodiments, when expressed in a T-cell, exhibit higher ligand-induced signal levels when the T-cell is activated, as compared to the ligand-induced signal level when the T-cell is not activated.

In some embodiments, the present disclosure provides, among other things, a new class of chimeric receptors containing a non-Notch transmembrane domain (TMD) including at least one γ-secretase site. It is believed that the modulation of TMD cleavage facilitates the optimization and/or improvement of the activity of the chimeric receptors disclosed herein, which in turn can be particularly useful in modulating cell activity and/or in treating diseases. In some embodiments, the chimeric receptors of the disclosure include non-Notch STS sequences, which allow the chimeric receptors disclosed herein to have a broad range of desired characteristics. For example, by altering the STS as described herein, and selecting different juxtamembrane domains, one can produce receptors having different noise levels (i.e., the basal level of receptor activation in the absence of the selected target ligand), different signal levels (i.e., the expression level obtained in the presence of the selected target ligand), and different signal:noise ratios.

In one aspect, provided herein are chimeric polypeptides including, from N-terminus to C-terminus: (a) an extracellular binding domain having a binding affinity for a selected ligand; (b) a linking sequence; (c) a transmembrane domain (TMD) having at least about 80% sequence identity to the transmembrane domain of a Type 1 transmembrane receptor and including one or more ligand-inducible proteolytic cleavage sites; and (d) an intracellular domain (ICD) including a transcriptional regulator, wherein binding of the selected ligand to the extracellular binding domain induces cleavage at the ligand-inducible proteolytic cleavage site between the transcriptional regulator and the linking sequence, and wherein the chimeric polypeptide does not include a sequence from a Notch receptor.

Non-limiting exemplary embodiments of the chimeric polypeptides provided herein include one or more of the following features: in some embodiments, the chimeric polypeptide further includes a stop-transfer sequence (STS) between the TMD and the ICD; in some embodiments, the TMD includes a polypeptide sequence having at least 80% sequence identity to a transmembrane domain from a Type 1 transmembrane receptor and includes a γ-secretase cleavage site; in some embodiments, the TMD includes a polypeptide sequence having at least 90% sequence identity to a transmembrane domain from a Type I transmembrane receptor and includes a γ-secretase cleavage site; in some embodiments, the TMD includes a polypeptide sequence having at least 95% sequence identity to a transmembrane domain from a Type I transmembrane receptor and includes a γ-secretase cleavage site. In some embodiments, the Type 1 transmembrane receptor is selected from the group consisting of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, IFNAR2, ILIR1, ILIR2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SORT1, SDC1, SDC2, TYR, TYRP1, DCT, VASN, FLT1, CDH5, PKHD1, NECTIN1, PCDHGC3, NRG1, LRPIB, CDH2, NRG2, PTPRK, SCN2B, Nradd, and PTPRM, and comprises a γ-secretase cleavage site. In some embodiments, the Type 1 transmembrane receptor is APP, CLSTN1, CLSTN2, EPCAM, KCNE3, NECTIN1, PCDHGC3, and PTPRF. In some embodiments, the chimeric polypeptides provided herein include a stop-transfer sequence (STS), and wherein the STS is heterologous to the TMD. In some embodiments, the STS includes a sequence having at least 70% sequence identity to a STS sequence from CSF1R, CXCL16, DAG1, GHR, PTPRF, AGER, KL, NRG1, LRPIB, Jag2, EPCAM, KCNE3, CDH2, NRG2, PTPRK, BTC, EPHA3, ILIR2, PTPRF, or PTPRM. In some embodiments, the STS comprises a sequence having at least 70% sequence identity to a STS sequence from CSF1R, DAG1, AGER, NRG1, KCNE3, PTPRF, or PTPRM.

In some embodiments, the extracellular domain includes an antigen-binding moiety capable of binding to a ligand on the surface of a cell. In some embodiments, the cell is a pathogenic cell. In some embodiments, the ligand includes a protein or a carbohydrate. In some embodiments, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, BCMA (CD269), ALPPL2, GFP, eGFP, and SIRPa.

In some embodiments, the ligand is selected from the group consisting of CD1, CDla, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE, Alkaline phosphatase, placental-like 2 (ALPPL2), B-cell maturation antigen (BCMA), Green Fluorescent Protein (GFP), Enhanced Green Fluorescent Protein (EGFP), and Signal regulatory protein α (SIRPα).

In some embodiments, the ligand is selected from cell surface receptors, adhesion proteins, integrins, mucins, lectins, tumor-associated antigens, and tumor-specific antigens. In some embodiments, the ligand is a tumor-associated antigen or a tumor-specific associated antigen. In some embodiments, the extracellular binding domain includes the ligand-binding portion of a receptor. In some embodiments, the antigen-binding moiety is selected from the group consisting of an antibody, a nanobody, a diabody, a triabody, or a minibody, a F(ab′)₂ fragment, a Fab fragment, a single chain variable fragment (scFv), a single domain antibody (sdAb), or a functional fragment thereof. In some embodiments, the antigen-binding moiety includes an scFv.

In some embodiments, the antigen-binding moiety is a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl. In some embodiments, the tumor-associated antigen is CD19, CEA, HER2, MUC1, CD20, BCMA, ALPPL2, or EGFR. In some embodiments, the tumor-associated antigen is CD19.

In some embodiments, the ligand-inducible proteolytic cleavage site is a γ-secretase cleavage site. In some embodiments, the transcriptional regulator includes a transcriptional activator, or a transcriptional repressor. In some embodiments, the ICD includes a nuclear localization sequence and a transcriptional regulator sequence selected from Gal4-VP16, Gal4-VP64, tetR-VP64, ZFHD1-VP64, Gal4-KRAB, and HAP1-VP16. In some embodiments, the chimeric polypeptides of the disclosure further include a signal sequence, a detectable label, a tumor-specific cleavage site, a disease-specific cleavage site, or a combination thereof.

In some embodiments, the chimeric polypeptides of the disclosure include an STS with an amino acid sequence having at least 80% sequence identity to SEQ ID NO: 89, 92, 108, 115, 138, 146, or 156.

In some embodiments, the hinge domain is capable of promoting oligomer formation of the chimeric polypeptide via intermolecular disulfide bonding. In some embodiments, the linking sequence includes an amino acid sequence having at least 80% sequence identity to a hinge domain derived from CD8α hinge domain, a CD28 hinge domain, a CD152 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof. In some embodiments, the hinge domain is derived from a CD8α hinge domain or a functional variant thereof. In some embodiments, the hinge domain comprises an amino acid sequence having at least 80% sequence identity to any one of SEQ ID NOS: 157-161.

In some embodiments, the TMD includes an amino acid sequence having at least 80% sequence identity to any one of SEQ ID NOS: 1-2, 6, 20, 43, 39, 54, and 64. In some embodiments, the TMD includes an amino acid sequence having at least 90% sequence identity to any one of SEQ ID NOS: 1-2, 6, 20, 43, 39, 54, and 64. In some embodiments, the TMD includes an amino acid sequence having at least 95% sequence identity to any one of SEQ ID NOS: 1-2, 6, 20, 43, 39, 54, and 64. In some embodiments, the TMD includes an amino acid sequence substantially identical to any one of SEQ ID NOS: 1-2, 6, 20, 43, 39, 54, and 64.

In some embodiments, the chimeric polypeptides of the disclosure include: (a) a linking sequence including an amino acid sequence having at least 80% sequence identity to any one of SEQ ID NOS: 157-161; (b) a TMD including an amino acid sequence having at least 80% sequence identity to any one of SEQ ID NO: 1-84, 178, 182-185, and 190-192; and (c) a STS including an amino acid sequence having at least 80% sequence identity to SEQ ID NO: 85-156 and 173.

In another aspect, provided herein are nucleic acids including a nucleotide sequence that encodes a chimeric polypeptide as disclosed herein. In some embodiments, the nucleotide sequence is incorporated into an expression cassette or an expression vector. In some embodiments, the expression vector is a viral vector. In some embodiments, the viral vector is a lentiviral vector, an adenovirus vector, an adeno-associated virus vector, or a retroviral vector. In some embodiments, the viral vector is a lentivival vector.

In another aspect, provided herein are recombinant cells including (a) a chimeric polypeptide as disclosed herein and/or (b) a recombinant nucleic acid as disclosed herein. In some embodiments, the cell is a mammalian cell. In some embodiments, the mammalian cell is an immune cell, a neuron, an epithelial cell, an endothelial cell, or a stem cell. In some embodiments, the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell. In some embodiments, the recombinant cell further includes: (a) a second chimeric polypeptide as disclosed herein; and/or (b) a second nucleic acid as disclosed herein; wherein the first chimeric polypeptide and the second chimeric polypeptide do not have the same sequence, and/or the first nucleic acid or the second nucleic acid do not have the same sequence. In some embodiments, the chimeric polypeptide modulates the expression and/or activity of the second chimeric polypeptide.

In some embodiments, the recombinant cell of the disclosure further includes an expression cassette encoding a protein of interest operably linked to a promoter, wherein expression of the protein is modulated by the transcriptional regulator encoded by the chimeric receptor. In some embodiments, the protein of interest is heterologous to the cell. In some embodiments, the promoter is GAL4. In some embodiments, the protein of interest is a cytokine, a cytotoxin, a chemokine, an immunomodulator, a pro-apoptotic factor, an anti-apoptotic factor, a hormone, a differentiation factor, a de-differentiation factor, an immune cell receptor, or a reporter.

In another aspect, further provided herein are cell cultures including at least one recombinant cell as disclosed herein and a culture medium.

In yet another aspect, provided herein are pharmaceutical compositions including a pharmaceutically acceptable carrier and one or more of the following: (a) a recombinant nucleic acid as disclosed herein, or (b) a recombinant cell as disclosed herein. In some embodiments, the disclosed pharmaceutical composition includes a recombinant nucleic acid as disclosed herein and a pharmaceutically acceptable carrier. In some embodiments, the recombinant nucleic acid is encapsulated in a viral capsid or a lipid nanoparticle (LNP).

In another aspect, provided herein are methods for modulating an activity of a cell, including: (a) providing a recombinant cell of the disclosure, and (b) contacting it with a selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. In some embodiments, the contacting is carried out in vivo, ex vivo, or in vitro. In some embodiments, the activity of the cell is selected from the group consisting of: expression of a selected gene of the cell, proliferation of the cell, apoptosis of the cell, non-apoptotic death of the cell, differentiation of the cell, de-differentiation of the cell, migration of the cell, secretion of a molecule from the cell, cellular adhesion of the cell, and cytolytic activity of the cell. In some embodiments, the released transcriptional regulator modulates expression of a gene product of the cell. In some embodiments, the released transcriptional regulator modulates expression of a heterologous gene product. In some embodiments, the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin-derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor. In some embodiments, the released transcriptional regulator modulates differentiation of the cell, and wherein the cell is an immune cell, a stem cell, a progenitor cell, or a precursor cell

Another aspect relates to methods for inhibiting an activity of a target cell in an individual, including administering to the individual an effective number of the recombinant cell of the disclosure, wherein the recombinant cell inhibits an activity of the target cell in the individual. In some embodiments, the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, provided herein are methods for treating a disease in an individual, the methods including a step of administering to the individual an effective number of the recombinant cell of the disclosure, wherein the recombinant cell treats the disease in the individual. In some embodiments, the treatment methods of the disclosure further include administering to the individual a second therapy. In some embodiments, the second therapy is selected from the group consisting of chemotherapy, radiotherapy, immunotherapy, hormonal therapy, or toxin therapy. In some embodiments, the first therapy and the second therapy are administered together, in the same composition or in separate compositions. In some embodiments, the first therapy and the second therapy are administered simultaneously. In some embodiments, the first therapy and the second therapy are administered sequentially. In some embodiments, the first therapy is administered before the second therapy. In some embodiments, the first therapy is administered after the second therapy. In some embodiments, the first therapy and the second therapy are administered in rotation.

In another aspect, provided herein are systems for modulating an activity of a cell, modulating an activity of a target cell, or treating a disease in an individual in need thereof, wherein the system includes one or more of: a chimeric polypeptide of the disclosure; a polynucleotide of the disclosure; a recombinant cell of the disclosure; or a pharmaceutical composition of the disclosure.

In another aspect, provided herein are methods for making a recombinant cell of the disclosure, including: (a) providing a cell capable of protein expression; and (b) contacting the provided cell with a recombinant nucleic acid of the disclosure. In some embodiments, the cell is obtained by leukapheresis performed on a sample obtained from a human subject or patient, and the cell is contacted ex vivo. In some embodiments, the recombinant nucleic acid is encapsulated in a viral capsid or a lipid nanoparticle (LNP).

In another aspect, provided herein is the use of one or more of: a chimeric polypeptide of the disclosure, a polynucleotide of the disclosure, a recombinant cell of the disclosure, or a pharmaceutical composition of the disclosure, for the treatment of a disease. In some embodiments, the disease is cancer.

In another aspect, provided herein is the use of one or more of: a chimeric polypeptide of the disclosure, a polynucleotide of the disclosure, a recombinant cell of the disclosure, or a pharmaceutical composition of the disclosure, in the manufacture of a medicament for the treatment of a disease.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1C schematically illustrate the experimental design and testing of exemplary non-Notch synthetic regulated intramembrane proteolysis receptors (SynRIPRs or SNIPRs) in accordance with some non-limiting embodiments of the disclosure. FIG. 1A schematically illustrates the modular assembly of an exemplary non-Notch SNIPR around a CLSTN2 core. FIGS. 1B-1C schematically summarize the results of JMD-based SNIPR tuning experiments. In these experiments, the reference receptor was an optimized CD8α Notch receptor (HingeNotch) composed of a truncated CD8α extracellular domain (ECD), a transmembrane domain (TMD) of human Notch1, and a C-terminal juxtamembrane domain (JMD) from Notch1. T cells expressing this anti-CD19 HingeNotch were able to activate a response element in the presence of CD19+ K562 sender cells. This activation was boosted when a co-expressed anti-ALPPL2 CAR is activated, providing a T cell activation stimulus. In each of these exemplary SynRIPRs, the extracellular binding domain comprises a single-chain antigen-binding fragment (scFv) having a binding affinity for a selected ligand, which in this example is the B-lymphocyte antigen CD19. As shown in FIGS. 1B-1C, assembly of a non-Notch SynRIPR using the CLSTN2 TMD and CLSTN2 JMD results in an inactive receptor, however the receptor function can be restored when the CLSTN2 JMD is replaced with a higher performing JMD, such as a JMD from KCNE3 or PTPRF.

FIG. 2 depicts the experimental design of a screen for TMDs and JMDs/STSs that can be deployed in the chimeric polypeptides and non-Notch SynRIPR described herein including modular assemblies of SynRIPRs with tunable transcriptional output. In these experiments, the Notch1 JMD (STS) was kept constant for TMD selection, whereas the Notch1 TMD was kept constant for JMD (STS) selection. T cells expressing high-performing “SynRIPR→BFP expression” circuits were co-incubated with sender cells for 48 hours. BFP output was measured using flow cytometry.

### DETAILED DESCRIPTION OF THE DISCLOSURE

The present disclosure generally relates to, among other things, novel chimeric polypeptides, including a new class of synthetic receptors that do not require sequence from Notch receptors. In particular, as described in greater detail below, systematic engineering efforts have illustrated that one can utilize (1) a ADAM protease cleavable extracellular domain (e.g., hinge domain from CD8), (2) a γ-secretase cleavable transmembrane domain (TMD), and (3) a stop-transfer-sequence to rationally build synthetic regulated intermembrane proteolysis receptors (SynRIPRs). As such, these receptors do not occur in nature. As described below, the chimeric polypeptides of the disclosure can be synthetic polypeptides, or can be engineered, designed, or modified so as to provide desired and/or improved properties, e.g., modulating transcription.

As described in greater detail below, these receptors can be used to target various therapeutically relevant cell types to sites of disease and regulate the therapeutic activity the cell in a controlled and localized fashion. These receptors can be linked to inducible transcriptional programs such that when the cell senses a disease site it will locally produce therapeutics such as antibodies, cytokines, toxins, and adjuvants. They are a highly versatile and programmable platform for cellular engineering, and essentially the production of any genetically encodable therapeutic can regulated by these receptors. As such, the SynRIPRs described herein could be potentially be used in any of the cell therapies for cancer, autoimmunity, or the regeneration of damaged tissue. In addition, these receptors are compact in size. Polynucleotides encoding the chimeric receptors provided herein can be made smaller than existing SynNotch-encoding polynucleotides, which enables the use of vectors having more limited capacity but otherwise more desirable characteristics, or the inclusion of additional elements that would otherwise be excluded vector capacity-related size constraints. The experimental data described herein demonstrate that advanced engineering efforts can now allow scientists to rationally design receptors that function like Notch receptors but share no sequence with the Notch proteins.

In some embodiments, the present disclosure provides a new class of chimeric receptors containing a non-Notch transmembrane domain (TMD) including at least one γ-secretase site. As described in greater detail below, cleavage of the TMD is an essential step in signaling mediated by these synthetic receptors. Without being bound to any particular theory, it is believed that modulation of TMD cleavage facilitates the optimization and/or improvement of the receptors disclosed herein, which in turn can be particularly useful in modulating cell activity, e.g., activating or inhibiting selected biosynthetic pathways and/or in treating diseases. In some embodiments, the receptors disclosed herein bind a target cell-surface displayed ligand, which triggers proteolytic cleavage of the receptors and release of a transcriptional regulator that modulates a custom transcriptional program in the cell.

In some embodiments, the chimeric receptors disclosed herein also include non-Notch STS sequences, which allow the new synthetic chimeric receptors of the disclosure to have a broad range of desired characteristics. For example, by altering the STS, and selecting different juxtamembrane domains, one can produce receptors having different noise levels (i.e., the basal level of receptor activation in the absence of the selected target ligand), different signal levels (i.e., the expression level obtained in the presence of the selected target ligand), and different signal:noise ratios.

Thus far, all functional SynNotch receptors have included sequence derived from Notch family members. As described in greater detail below, to demonstrate the versatility of the modular receptor assembly approach, experiments have been designed to engineer a ligand-activated receptor from the ground up based on receptor components that were completely devoid of Notch sequences. It was observed that although all receptors expressed at high levels, the otherwise-active Alcadein-gamma (CSTLN2) TMD did not function with its cognate, but inactive, STS/JMD sequence. Remarkably, it was observed that receptor signaling was restored by replacing the CSTLN2 JMD with a more active one from KCNE3 (RSRKVDKR; SEQ ID: NO: 146) or PTPRF (KRKRTH; SEQ ID: NO: 108), suggesting that the original CLSTN2 JMD is functionally limiting. The experimental results described herein illustrate the ability to build new functional synthetic regulated intramembrane proteolysis receptors from a set of functional parts, which in turns demonstrates that synthetic biology approach of receptor assembly is capable of generating receptors that function like Notch receptors but can be rationally customized in their activity.

The disclosure also provides compositions and methods useful for producing such receptors, nucleic acids encoding same, host cells genetically modified with the nucleic acids, as well as methods for modulating an activity of a cell and/or for the treatment of various diseases such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used interchangeably herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the original cell or cell line.

As used herein, the term “construct” is intended to mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, either capable of genomic integration or autonomous replication, including a nucleic acid molecule where one or more nucleic acid sequences has been linked in a functionally operative manner, e.g., operably linked.

The term “operably linked”, as used herein, denotes a physical or functional linkage between two or more elements, e.g., polypeptide sequences or polynucleotide sequences, which permits them to operate in their intended fashion.

The term “heterologous”, refers to nucleic acid sequences or amino acid sequences operably linked or otherwise joined to one another in a nucleic acid construct or chimeric polypeptide that are not operably linked or are not contiguous to each other in nature.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI website at ncbi.nlm.nih.gov/BLAST. This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant cells, proteins, and nucleic acids include cells, proteins, and nucleic acids produced by laboratory methods. Recombinant proteins can include amino acid residues not found within the native (non-recombinant or wild-type) form of the protein or can be include amino acid residues that have been modified, e.g., labeled. The term recombinant can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, and unless otherwise specified, a “therapeutically effective amount” of an agent is an amount or number sufficient to provide a therapeutic benefit in the treatment or management of a health condition or disease, e.g., cancer, or to delay or minimize one or more symptoms associated with the condition or disease. A therapeutically effective amount or number of a therapeutic agent means an amount or number of the therapeutic agent, alone or in combination with other therapeutic agents, which provides a therapeutic benefit in the treatment or management of the condition or disease. The term “therapeutically effective amount” can encompass an amount that improves overall therapy, reduces or avoids symptoms or causes of the condition or disease, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount or number of a therapeutic agent including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” can be a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

As described in greater detail below, the present disclosure provides a new class of chimeric polypeptide receptors engineered to modulate transcriptional regulation in a ligand-dependent manner having multiple advantages over existing synthetic receptors. In particular, some embodiments of the disclosure provides synthetic receptors that do not require sequence from Notch receptors. In some embodiments, inclusion of a heterologous TMD provides the new synthetic receptors with a variety of improved expression characteristics, thus expanding the palette of available receptors available to the cellular engineer. In some embodiments, the synthetic receptors disclosed herein also include non-Notch STS sequences, which allow the new synthetic receptors to have a broad range of desired characteristics. In addition, as discussed above, these new synthetic receptors are compact in size, such that polynucleotides encoding these synthetic receptors can be made smaller than existing SynNotch-encoding polynucleotides, which enables the use of vectors having more limited capacity but otherwise more desirable characteristics, or the inclusion of additional elements that would otherwise be excluded vector capacity-related size constraints.

As illustrated below, the ability to build new functional synthetic regulated intramembrane proteolysis receptors from a set of functional parts shows that synthetic biology approaches of receptor assembly are capable of generating receptors that function like naturally occurring receptors, such as Notch receptors, but can be rationally to customize their activity.

One skilled in the art will understand that the chimeric polypeptide receptors disclosed herein facilitate amplified activation under certain specific cellular and environmental contexts. This type of feedback on the receptor activity is a new feature that can be exploited to enhance and tune the expression of therapeutic payloads by engineered cells. As described in the Examples herein, chimeric polypeptide receptors have been tested and validated in primary human T cells. Without being bound to any particular theory, it is contemplated that these new receptors show similar performance in other mammalian cells. In addition, one of ordinary skill in the art, upon reviewing the present disclosure, will appreciate that the synthetic receptors disclosed herein may be engineered into various immune cell types for enhanced discrimination and elimination of tumors, or in engineered cells for tunable control of autoimmunity and tissue regeneration. Accordingly, engineered cells, such as immune cells engineered to express one of more of the chimeric receptors disclosed herein, are also within the scope of the disclosure.

### Chimeric Polypeptides

In one aspect, provided herein are a variety of novel, non-naturally occurring chimeric polypeptides engineered to modulate transcriptional regulation in a ligand-dependent manner. These new receptors generally include (1) a γ-secretase cleavable transmembrane domain (TMD), and (2) a stop-transfer-sequence. Inclusion of heterologous STS and TMD sequences facilitates the optimization and/or improvement of the new chimeric receptors disclosed herein, which in turn is useful in modulating cell activity and/or in treating diseases. In some embodiments, the receptors provided herein bind a target cell-surface displayed ligand, triggering proteolytic cleavage of the receptors and releasing a transcriptional regulator that modulates a custom transcriptional program in the cell.

In some embodiments, provided herein is a chimeric polypeptide including, from N-terminus to C-terminus: (a) an extracellular binding domain having a binding affinity for a selected ligand; (b) a linking sequence; (c) a TMD having at least about 80% sequence identity to the transmembrane domain of a Type 1 transmembrane receptor and including one or more ligand-inducible proteolytic cleavage sites; and (d) an ICD including a transcriptional regulator, wherein binding of the selected ligand to the extracellular binding domain induces cleavage at the ligand-inducible proteolytic cleavage site between the transcriptional regulator and the linking sequence, and wherein the chimeric polypeptide does not include a sequence from a Notch receptor.

### Extracellular Domain (ECD)

In some embodiments, the ECD of the chimeric polypeptides and chimeric receptors (e.g., SynRIPRs) disclosed herein has a binding affinity for one or more target ligands. In principle, there are no particular limitations with regard to suitable ligands that may be targeted by the chimeric polypeptides provided herein. In some embodiments, the target ligand is a cell-surface ligand. Non-limiting examples of suitable cell-surface ligands include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate.

In some embodiments, the extracellular domain includes the ligand-binding portion of a receptor. In some embodiments, the extracellular domain includes an antigen-binding moiety that binds to one or more target antigens. In some embodiments, the ECD includes an antigen-binding moiety capable of binding to one or more antigens on the surface of a cell (e.g., cell surface antigens). In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For example, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety includes an scFv.

The antigen-binding moiety can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that binds the antigen with high affinity and does not significantly bind other unrelated antigens.

A skilled artisan can select an extracellular domain based on the desired localization or function of a cell that is genetically modified to express a chimeric polypeptide or SynRIPR of the present disclosure. For example, the extracellular domain can be selected to target a receptor-expressing cell to estrogen-dependent breast cancer cells. In some embodiments, the extracellular domain of the disclosed chimeric polypeptide receptors (e.g., SynRIPRs) is capable of binding a tumor-associated antigen (TAA) or a tumor-specific antigen (TSA). TAAs include a molecule, such as, for example, a protein, present on tumor cells and on a sub-population of normal cells, or on many normal cells, but at much lower concentration than on tumor cells. Examples include, without limitation, CEA, AFP, HER2, CTAGIB and MAGEA1. In contrast, TSAs generally include a molecule, such as, e.g., a protein, present on tumor cells but not expressed on normal cells. Examples include, without limitation, oncoviral antigens and mutated proteins (also known as neoantigens).

In some cases, the antigen-binding moiety is specific for an epitope present in an antigen that is expressed by a tumor cell, i.e., a tumor-associated antigen or a tumor-specific antigen. The tumor-associated or tumor-specific antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, and the like. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Generally, there are no particular limitations with regard to suitable surface antigens that may be targeted by the chimeric polypeptide receptors disclosed herein. Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein a (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7-H3), IL-13Rα1, IL-13Rα2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), and mesothelin.

Further non-limiting examples of suitable antigens include PAP (prostatic acid phosphatase), prostate stem cell antigen (PSCA), prostein, NKG2D, TARP (T cell receptor γ alternate reading frame protein), Trp-p8, STEAPI (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), Ral-B, GPC2, CD276 (B7-H3), or IL-13Rα. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

Additional antigens that can be suitable for the chimeric polypeptide receptors disclosed herein include, but are not limited to GPC2, human epidermal growth factor receptor 2 (Her2/neu), CD276 (B7-H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional suitable antigens include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (one subunit of the heterodimeric IL-2 receptor), CD3, CD4, CD5, IFNα, IFNγ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNFα, and VEGF-A.

Further antigens suitable for the chimeric polypeptides and SynRIPRs disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAPI (six-transmembrane epithelial antigen of the prostate-1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

In some embodiments, antigens suitable for targeting by the chimeric polypeptides and chimeric receptors disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the extracellular domain is specific for a cell surface target, where non-limiting examples of cell surface targets include CD19, CD30, Her2, CD22, ENPP3, EGFR, CD20, CD52, CD11α, and α-integrin. In some embodiments, the chimeric polypeptides and SynRIPRs disclosed herein include an extracellular domain having an antigen-binding moiety that binds CD19, CEA, HER2, MUC1, CD20, or EGFR. In some embodiments, the chimeric polypeptides and SynRIPRs disclosed herein include an extracellular domain including an antigen-binding moiety that binds CD19.

### Linking Sequence

As described above, the chimeric polypeptides and receptors of the disclosure include a linking sequence disposed between the extracellular ligand-binding domain (ECD) and the TMD.

In some embodiments, the linking sequence of the chimeric receptors has at least about 80% amino acid sequence identity to a polypeptide hinge domain. Without being bound to any particular theory, the hinge domain of the chimeric polypeptides and SynRIPRs disclosed herein may serve several functions, including controlling flexibility and rigidity of the chimeric polypeptides and SynRIPRs, which in turn can affect antigen binding and signal transduction. The length of the hinge domain can be tuned to enhance the SynRIPRs' ability to reach antigens in the space between a SynRIPR and a target cell. The hinge domain can also be tuned to mediate dimerization. Care should be taken in selecting a suitable hinge domain. For example, shorter hinge domain may be more rigid and more effective at transducing signal once engaged on target antigen but may not reach target that are more membrane proximal or otherwise specially constrained. In some embodiments, the hinge domain is capable of promoting oligomer formation of the chimeric polypeptide via intermolecular disulfide bonding. In these instances, within the chimeric polypeptides and SynRIPRs disclosed herein, the hinge domain generally includes a flexible oligo- or polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40).

Hinge polypeptide sequences suitable for the compositions and methods of the disclosure can be naturally-occurring hinge polypeptide sequences (e.g., those from naturally-occurring immunoglobulins). Alternatively, a hinge polypeptide sequence can be a synthetic sequence that corresponds to a naturally-occurring hinge polypeptide sequence, or can be an entirely synthetic hinge sequence, or can be engineered, designed, or modified to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs. Additional information in this regard can be found in, for example, a recent review by Vidarsson G. et al., Frontiers Immunol. Oct. 20, 2014, which is hereby incorporated by reference in its entirety.

Accordingly, in some embodiments, the hinge domain includes a hinge polypeptide sequence derived from an IgG1 hinge domain or a functional variant thereof. In some embodiments, the hinge domain includes a hinge polypeptide sequence derived from an IgG2 hinge domain or a functional variant thereof. In some embodiments, the hinge domain includes a hinge polypeptide sequence derived from an IgG3 hinge domain or a functional variant thereof. In some embodiments, the hinge domain includes a hinge polypeptide sequence derived from an IgG4 hinge domain or a functional variant thereof. In some embodiments, the hinge domain includes a hinge polypeptide sequence derived from an IgA hinge domain or a functional variant thereof. In some embodiments, the hinge domain includes a hinge polypeptide sequence derived from an IgD hinge domain or a functional variant thereof.

Additional hinge polypeptide sequences suitable for the compositions and methods disclosed herein include, but are not limited to, hinge polypeptide sequences derived from a CD8α hinge domain, a CD28 hinge domain, a CD152 hinge domain, a PD-1 hinge domain, a CTLA4 hinge domain, an OX40 hinge domain, an FcγRIIIα hinge domain, and functional variants thereof. In some embodiments, the hinge domain includes a hinge polypeptide sequence derived from a CD8α hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

In principle, there are no particular limitations to the length and/or amino acid composition of the hinge domain other than it confers flexibility and the capacity for oligomerization. However, one skilled in the art will readily appreciate that the length and amino acid composition of the hinge polypeptide sequence can be optimized to vary the orientation and/or proximity of the ECD and the TMD relative to one another, as well as of the chimeric polypeptide monomers to one another, to achieve a desired activity of the chimeric polypeptide of the disclosure. In some embodiments, any single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a hinge domain. In some embodiments, the hinge domain includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the hinge domain includes about 1 to 10, about 5 to 15, about 10 to 20, about 15 to 25, about 20 to 40, about 30 to 50, about 40 to 60, about 50 to 70 amino acid residues. In some embodiments, the hinge domain includes about 40 to 70, about 50 to 80, about 60 to 80, about 70 to 90, or about 80 to 100 amino acid residues. In some embodiments, the hinge domain includes about 1 to 10, about 5 to 15, about 10 to 20, about 15 to 25 amino acid residues.

In some embodiments, the linking sequence comprises an amino acid sequence having at least 80% sequence identity to a hinge domain derived from CD8α hinge domain, a CD28 hinge domain, a CD152 hinge domain, a PD-1 hinge domain, a CTLA4 hinge domain, an OX40 hinge domain, an IgGI hinge domain, an IgG2 hinge domain, an IgG3 hinge domain, and an IgG4 hinge domain, or a functional variant of any thereof.

In some embodiments, the hinge domain is derived from a CD8α hinge domain or a functional variant thereof. In some embodiments, the hinge domain comprises an amino acid sequence having at least 80% sequence identity to any one of SEQ ID NOS: 157-161.

In some embodiments, the linking sequence comprises or consists of an amino acid sequence having at least about 80% sequence identity, such as, at least 80%, at least 85%, at least 90%, at least 95%, at least 96%, at least 97%, at least 98%, or about 99% sequence identity to a sequence set forth in SEQ ID NOS: 157-161 in the Sequence Listing. In some embodiments, the linking sequence comprises or consists of an amino acid sequence having at least about 80% sequence identity to a sequence set forth in SEQ ID NOS: 157-161. In some embodiments, the linking sequence comprises or consists of an amino acid sequence having at least about 90% sequence identity to a sequence set forth in SEQ ID NOS: 157-161. In some embodiments, the linking sequence comprises or consists of an amino acid sequence having at least about 95% sequence identity to a sequence set forth in SEQ ID NOS: 157-161. In some embodiments, the linking sequence comprises or consists of an amino acid sequence having about 100% sequence identity to a sequence set forth in SEQ ID NOS: 157-161. In some embodiments, the linking sequence comprises or consists of an amino acid sequence selected from the group consisting of SEQ ID NOS: 157-161, wherein one, two, three, four, or five of the amino acid residues in any one of SEQ ID NO: 157-161 is/are substituted by a different amino acid residues.

In some embodiments, one or more domains of a ROBO (Roundabout) cell surface receptor are incorporated into the linking sequence of the chimeric receptors of the present disclosure. ROBO receptors release a nuclear transcription factor domain following ligand-induced cleavage of the extracellular portion of the receptor by the ADAM10 MMP and γ-secretase. However, ROBO does not contain a LIN/Notch domain, EGF-like repeats, or an HD domain. Additionally, the primary ligand for ROBO is a soluble protein (SLIT). Mammals have four ROBO receptors: ROBO1-3 have five immunoglobulin-like (Ig) domains, three fibronectin (Fn) repeats, and a transmembrane domain linked to an intracellular domain. ROBO4 has only two Ig domains, and two Fn domains. Additional information in this regard can be found in, for example, H. Blockus et al., Development (2016) 143:3037-44, which is hereby incorporated by reference.

In some embodiments, the linking sequence of the chimeric receptors as disclosed herein includes at least one fibronectin (Fn) repeat derived from a ROBO receptor. The linking sequence of the disclosed chimeric receptors can contain 1, 2, 3, 4, or 5 Fn repeats. In some embodiments, the l linking sequence of the chimeric receptors includes about 1 to about 5 Fn repeats, about 1 to about 3 Fn repeats, or about 2 to about 3 Fn repeats. In some embodiments, the linking sequence of the chimeric receptors has at least about 80% amino acid sequence identity to a ROBO1 JMD including at least one fibronectin repeat.

In some embodiments, the linking sequence of the chimeric receptors has a polypeptide sequence of about 4 to about 40 amino acid residues in length (e.g., 4, 5, 6, 7, 8, 9, 10, 11, 12, 13, 14, 15, 16, 17, 18, 19, 20, or more amino acid residues). In some embodiments, the length and amino acid composition of the linking sequence can be optimized to vary the orientation and/or proximity of the ECD and the TMD relative to one another to achieve a desired activity of the chimeric polypeptide of the disclosure. In some embodiments, the linking sequence of the chimeric receptors has a polypeptide sequence of about 4 to about 40, about 5 to about 30, about 10 to about 20, about 10 to about 30, about 10 to about 40, about 5 to about 20, about 5 to about 40, or about 20 to about 40 amino acid residues in length.

### Transmembrane Domain (TMD)

As described above, the chimeric receptors of the disclosure include a transmembrane domain having at least about 80% sequence identity to the TMD of a Type 1 transmembrane receptor and comprising one or more ligand-inducible proteolytic cleavage sites.

Examples of proteolytic cleavage sites suitable for the compositions and methods disclosed herein include, but are not limited to, a metalloproteinase cleavage site for a matrix metalloproteinase (MMP) selected from collagenase-1, -2, and -3 (MMP-1, - 8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue such as Leu, Ile, Val, Phe, Trp, Tyr, Val, Met, and Pro) (SEQ ID NO: 165), e.g., Pro-X-X-Hy-(Ser/Thr) (SEQ ID NO: 166), e.g., Pro-Leu/Gln-Gly-Met-Thr-Ser (SEQ ID NO: 167) or Pro-Leu/Gln-Gly-Met-Thr (SEQ ID NO: 168. Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg (SEQ ID NO: 169). Another example of a protease cleavage site that can be included in a proteolytically cleavable linker is a tobacco etch virus (TEV) protease cleavage site, e.g., Glu-Asn-Leu-Tyr-Thr-Gln-Ser (SEQ ID NO: 170), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 171), where cleavage occurs after the lysine residue. Another example of a protease cleavage site that can be included in a proteolytically cleavable linker is a thrombin cleavage site, e.g., Leu-Val-Pro-Arg (SEQ ID NO: 172). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrix metalloproteinase (MT-MMP), stromelysin 3 (or MMP-11), thermolysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the SynRIPR of the disclosure is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor comprising at least one γ-secretase cleavage site. Detailed description of the structure and function of the γ-secretase complex as well as its substrate proteins, including amyloid precursor protein (APP), can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors ILIR1, ILIR2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SORT1, SDC1, SDC2, SPN, TYR, TYRP1, DCT, VASN, FLT1, CDH5, PKHD1, NECTIN1, PCDHGC3, NRG1, LRPIB, CDH2, NRG2, PTPRK, SCN2B, Nradd, and PTPRM. In some embodiments, the TMD of the chimeric polypeptides or SynRIPRs of the disclosure is a TMD derived from the TMD of APP, CLSTN1, CLSTN2, EPCAM, KCNE3, NECTIN1, PCDHGC3, and PTPRF. In some embodiments, the TMD of the chimeric polypeptides or SynRIPRs of the disclosure is a TMD derived from the TMD of a member of the calsyntenin family, such as, alcadein alpha and alcadein gamma.

Accordingly, in some embodiments, the transmembrane domain includes an amino acid sequence exhibiting at least 70%, at least 75%, at least 80%, at least 85%, at least 90%, at least 95%, at least 96%, at least 97%, at least 98%, at least 99% sequence identity to a polypeptide sequence having at least about 70% sequence identity to a transmembrane domain from a Type 1 transmembrane receptor that comprises a γ-secretase cleavage site. In some embodiments, the transmembrane domain includes an amino acid sequence exhibiting at least 70% sequence identity to a transmembrane domain from a Type 1 transmembrane receptor that comprises a γ-secretase cleavage site. In some embodiments, the transmembrane domain includes an amino acid sequence exhibiting at least about 80% sequence identity to a transmembrane domain from a Type 1 transmembrane receptor that comprises a γ-secretase cleavage site. In some embodiments, the transmembrane domain includes an amino acid sequence exhibiting at least about 90% sequence identity to a transmembrane domain from a Type 1 transmembrane receptor that comprises a γ-secretase cleavage site. In some embodiments, the transmembrane domain includes an amino acid sequence exhibiting at least about 95% sequence identity to a transmembrane domain from a Type 1 transmembrane receptor that comprises a γ-secretase cleavage site. In some embodiments, the Type 1 transmembrane receptor is selected from the group consisting of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, IFNAR2, ILIR1, ILIR2, IL6R, INSR, ERNI, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SORT1, SDC1, SDC2, TYR, TYRP1, DCT, VASN, FLT1, CDH5, PKHD1, NECTIN1, PCDHGC3, NRG1, LRPIB, CDH2, NRG2, PTPRK, SCN2B, Nradd, and PTPRM. In some embodiments, the TMD includes an amino acid sequence exhibiting at least about 70%, at least about 75%, at least about 80%, at least about 85%, at least about 90%, at least about 95%, at least about 96%, at least about 97%, at least about 98%, or at least about 99% sequence identity to any one of SEQ ID NOS: 1-84, 178, 182-185, and 190-192 in the Sequence Listing. In some embodiments, the transmembrane domain includes an amino acid sequence having at least about 90% sequence identity to SEQ ID NOS: 1-84, 178, 182-185, and 190-192. In some embodiments, the transmembrane domain includes an amino acid sequence having at least about 95% sequence identity to SEQ ID NOS: 1-88. In some embodiments, the transmembrane domain includes an amino acid sequence having about 100% sequence identity to SEQ ID NOS: 1-84, 178, 182-185, and 190-192. In some embodiments, the transmembrane domain includes the amino acid sequence of SEQ ID NO: 1-84, 178, 182-185, and 190-192, wherein one, two, three, four, or five of the amino acid residues in the amino acid sequence is/are substituted by a different amino acid residue.

In some embodiments, the amino acid substitution(s) within the TMD includes one or more substitutions within a “GV” motif of the TMD. In some embodiments, at least one of such substitution(s) comprises a substitution to alanine. For example, one, two, three, four, five, or more of the amino acid residues of the sequence IATVVIIISVCMLVFVVAMGVY (SEQ ID NO: 2), as well as any one of sequences as set forth in SEQ ID NOS: 1, 6, 20, 43, 39, 54, and 64, may be substituted by a different amino acid residue. In some embodiments, the amino acid residue at position 18 and/or 19 of the “GV” motif within SEQ ID NO: 2 is substituted by a different amino acid residue. In some embodiments, the glycine residue at position 20 of SEQ ID NO: 2 is substituted by a different amino acid residue. In some embodiments, the valine residue at position 21 of SEQ ID NO: 2 is substituted by a different amino acid residue. In some embodiments, the transmembrane domain comprises an amino acid sequence having a sequence corresponding to SEQ ID NO: 2 with a mutation at the position corresponding to position 18 of SEQ ID NO: 20, such as G18A mutations. In some embodiments, the transmembrane domain comprises an amino acid sequence having a sequence corresponding to SEQ ID NO: 2 with a mutation at the position corresponding to position 21 of SEQ ID NO: 57, such as V19A mutations.

### Stop-Transfer Sequence (STS)

In some embodiments, the chimeric receptors of the disclosure include a stop-transfer sequence (STS), which is essentially a highly-charged domain located between the TMD and the ICD. Without being bound to any particular theory, such a highly-charged domain disposed between the TMD and the ICD prevents the ICD from entering the membrane. In principle, there are no particular limitations to the length and/or amino acid composition of the STS. In some embodiments, any arbitrary single-chain peptide comprising about 1 to about 40 amino acid residues (e.g., 1, 2, 3, 4, 5, 6, 7, 8, 9, 10, 11, 12, 13, 14, 15, 16, 17, 18, 19, 20, or more amino acid residues) can be used as an STS. In some embodiments, the STS includes about 1 to 15, about 5 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 1 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues.

In some embodiments, the STS includes a sequence having at least about 80% sequence identity, such as, at least 80%, at least 85%, at least 90%, at least 95%, at least 96%, at least 97%, at least 98%, or 99% sequence identity to the STS domain of a Type 1 receptor. In some embodiments, the STS includes an amino acid sequence having at least 90% sequence identity to the STS domain of a Type 1 receptor. In some embodiments, the STS includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from CSF1R, CXCL16, DAG1, GHR, PTPRF, AGER, KL, NRG1, LRPIB, Jag2, EPCAM, KCNE3, CDH2, NRG2, PTPRK, BTC, EPHA3, IL1R2, PTPRF, or PTPRM. In some embodiments, the STS includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from CSF1R, DAG1, AGER, NRG1, KCNE3, PTPRF, or PTPRM. In some embodiments, the STS includes a sequence comprising only Lys (K) or Arg (R) in the first 4 residues. In some embodiments, the STS includes one, two, three, four, five, or more basic residues. In some embodiments, the STS includes five, four, three, two, one, or zero aromatic residues or residues with hydrophobic and/or bulky side chains.

In some embodiments, the STS includes a sequence having at least 80% sequence identity, such as, at least 80%, at least 85%, at least 90%, at least 95%, at least 96%, at least 97%, at least 98%, or 99% sequence identity to SEQ ID NO: 85-156 and 173 in the Sequence Listing. In some embodiments, the STS includes an amino acid sequence having at least 90% sequence identity to SEQ ID NO: 89, 92, 108, 115, 138, 146, and 156. In some embodiments, the STS includes an amino acid sequence having at least 95% sequence identity to SEQ ID NOS: 89, 92, 108, 115, 138, 146, and 156. In some embodiments, the STS includes an amino acid sequence having at least 100% sequence identity to SEQ ID NO: 89, 92, 108, 115, 138, 146, and 156. In some embodiments, the STS includes an amino acid sequence having at least 90% or at least 95% sequence identity to SEQ ID NO: 146. In some embodiments, the STS includes an amino acid sequence having at least 95% sequence identity to SEQ ID NO: 108. In some embodiments, the STS includes the amino acid sequence of any one of SEQ ID NOS: 89, 92, 108, 115, 138, 146, and 156, wherein one, two, three, four, or five of the amino acid residues in the amino acid sequence is/are substituted by a different amino acid residue.

### Intracellular Domain (ICD)

The chimeric receptor of the disclosure further comprises an intracellular domain (ICD) comprising a transcriptional regulator. The transcriptional regulator is a biochemical element that acts to activate or repress the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. In some embodiments, the transcriptional regulator directly regulates expression of one or more genes involved in differentiation of the cell. In some embodiments, the transcriptional regulator indirectly modulates expression of one or more genes involved in differentiation of the cell by modulating the expression of a second transcription factor which in turn modulates expression of one or more genes involved in differentiation of the cell. It will be understood by a skilled artisan that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcriptional regulator can further include a nuclear localization signal. In some embodiments, the transcriptional regulator is selected from Gal4-VP16, Gal4-VP64, tetR-VP64, ZFHD1-VP64, Gal4-KRAB, and HAP1-VP16. In some embodiments, the transcriptional regulator is Gal4-VP64.

### Additional Domains

In some embodiments, the extracellular domains located N-terminally to the TMD can further include an additional domain, for example a membrane localization signal such as a CD8α signal, a detectable marker such as a myc tag or his tag, and the like. In some embodiments, the chimeric receptors further comprise one or more additional proteolytic cleavage sites. In some embodiments, the chimeric receptors do not comprise an additional proteolytic cleavage site. In some embodiments, the chimeric receptors further comprise one or more glycosylation sites. In some embodiments, the chimeric receptors do not comprise a glycosylation site. In some embodiments, the chimeric receptors do not comprise a hinge domain for promoting oligomerization of the chimeric polypeptide via intermolecular disulfide bonding.

In some embodiments, the chimeric receptors further comprise elements of the highly conserved ROBO cell surface receptors. In some embodiments, the chimeric receptors disclosed comprise, from N-terminus to C-terminus: (a) an extracellular ligand binding domain having a binding affinity for a selected ligand; (b) a ROBO domain comprising a fibronectin repeat; (c) a TMD including one or more ligand-inducible proteolytic cleavage sites; and (d) an ICD including a transcriptional regulator, wherein binding of the selected ligand to the extracellular binding domain induces cleavage at the ligand-inducible proteolytic cleavage site in the TMD, and wherein the chimeric polypeptide does not include a sequence from a Notch receptor.

In some embodiments, the chimeric receptors further comprise an extracellular oligomerization domain (e.g., a hinge domain) to promote oligomerization, e.g., dimerization, trimerization, or higher order multimers of the chimeric receptor. In some embodiments, the chimeric receptors disclosed comprise, from N-terminus to C-terminus: (a) an extracellular ligand binding domain having a binding affinity for a selected ligand; (b) a hinge domain; (c) a TMD including one or more ligand-inducible proteolytic cleavage sites; and (d) an ICD including a transcriptional regulator, wherein binding of the selected ligand to the extracellular binding domain induces cleavage at the ligand-inducible proteolytic cleavage site in the TMD, and wherein the chimeric polypeptide does not include a sequence from a Notch receptor.

In some embodiments, the hinge domain includes polypeptide motifs capable of promoting oligomer formation of the chimeric polypeptide via intermolecular disulfide bonding. In some embodiments, a chimeric receptor disclosed herein includes, from N-terminus to C-terminus: (a) an extracellular ligand binding domain having a binding affinity for a selected ligand; (b) a hinge domain capable of promoting oligomer formation of the chimeric polypeptide via intermolecular disulfide bonding; (c) a TMD comprising one or more ligand-inducible proteolytic cleavage sites; and (d) an ICD comprising a transcriptional regulator, wherein binding of the selected ligand to the extracellular binding domain induces cleavage at a ligand-inducible proteolytic cleavage sites in the TMD, and wherein the chimeric polypeptide does not comprise a sequence from a Notch receptor.

Chimeric receptors of the present disclosure can be chimeric polypeptides of any length, including chimeric polypeptides that are generally between about 100 amino acids (aa) to about 1000 aa, e.g., from about 100 aa to about 200 aa, from about 150 aa to about 250 aa, from about 200 aa to about 300 aa, from about 250 aa to about 350 aa, from about 300 aa to about 400 aa, from about 350 aa to about 450 aa, from about 400 aa to about 500 aa in length. In some embodiments, the disclosed chimeric polypeptides are generally between about 400 aa to about 450 aa, from about 450 aa to about 500 aa, from about 500 aa to about 550 aa, from about 550 aa to about 600 aa, from about 600 aa to about 650 aa, from about 650 aa to about 700 aa, from about 700 aa to about 750 aa, from about 750 aa to about 800 aa, from about 800 aa to about 850 aa, from about 850 aa to about 900 aa, from about 900 aa to about 950 aa, or from about 950 aa to about 1000 aa in length. In some cases, the chimeric polypeptides of the present disclosure have a length of from about 300 aa to about 400 aa. In some cases, the chimeric polypeptides of the present disclosure have a length of from 300 aa to 350 aa. In some cases, the chimeric polypeptides of the present disclosure have a length of from 300 aa to 325 aa. In some cases, the chimeric polypeptides of the present disclosure have a length of from 350 aa to 400 aa. In some cases, the chimeric polypeptides of the present disclosure have a length of from 750 aa to 850 aa.

In some embodiments, the chimeric receptors of the disclosure include: (a) an extracellular ligand-binding domain (b) a linking sequence including an amino acid sequence having at least about 80%, 90%, 95%, 96%, 97, 98%, 99%, or 100% sequence identity to any one of SEQ ID NOS: 157-161; (c) a TMD including an amino acid sequence having at least about 80%, 90%, 95%, 96%, 97, 98%, 99%, or 100% sequence identity to any one of SEQ ID NOS: 1-84, 178, 182-185, and 190-192; (d) an STS including an amino acid sequence having at about least 80%, 90%, 95%, 96%, 97, 98%, 99%, or 100% sequence identity to any one of SEQ ID NOS: 89, 92, 108, 115, 138, 146, and 156; and (e) an ICD comprising a transcriptional regulator. In some embodiments, the chimeric receptor of the disclosure include: (a) a linking sequence including an amino acid sequence having at least about 90% sequence identity to any one of SEQ ID NOS: 157-161; (b) a TMD including an amino acid sequence having at least about 90% sequence identity to any one of SEQ ID NOS: 1-84, 178, 182-185, and 190-192; and (c) an STS including an amino acid sequence having at least about 90% sequence identity to any one of SEQ ID NO: 89, 92, 108, 115, 138, 146, and 156. In some embodiments, the chimeric receptors of the disclosure include: (a) a linking sequence including an amino acid sequence having at least about 95% sequence identity to any one of SEQ ID NO: 157-161; (b) a transmembrane domain including an amino acid sequence having at least about 95% sequence identity to any one of SEQ ID NO: 1-84, 178, 182-185, and 190-192; and (c) an STS including an amino acid sequence having at least about 95% sequence identity to any one of SEQ ID NOS: 89, 92, 108, 115, 138, 146, and 156.

In some embodiments, the chimeric polypeptides of the disclosure include an amino acid sequence having at least about 80%, 90%, 95%, 96%, 97, 98%, 99%, or 100% sequence identity to a chimeric receptor disclosed herein, e.g., those described in Table 1. Accordingly, in some embodiments, the chimeric polypeptides of the disclosure include an amino acid sequence having at least about 80%, 90%, 95%, 96%, 97, 98%, 99%, or 100% sequence identity to a sequence selected from the group consisting of SEQ ID NOs: 197-248 in the Sequence Listing. In some embodiments, the chimeric polypeptides of the disclosure include an amino acid sequence having at least about 90% sequence identity to a sequence selected from the group consisting of SEQ ID NOs: 197-248. In some embodiments, chimeric polypeptides of the disclosure include an amino acid sequence having at least about 95% sequence identity to a sequence selected from the group consisting of SEQ ID NOs: 197-248. In some embodiments, the chimeric polypeptides of the disclosure include an amino acid sequence having at least about 100% sequence identity to a sequence selected from the group consisting of SEQ ID NOs: 197-248. In some embodiments, the chimeric polypeptides of the disclosure include an amino acid sequence having at least about 100% sequence identity to a sequence selected from the group consisting of SEQ ID NOs: 197-248, wherein one, two, three, four, or five of the amino acid residues in the amino acid sequence is/are substituted by a different amino acid residue.

### Nucleic Acid Molecules

In one aspect, some embodiments of the disclosure relate to nucleic acid molecules comprising nucleotide sequences encoding the chimeric polypeptides and Notch receptors of the disclosure, including expression cassettes, and expression vectors containing these nucleic acid molecules operably linked to heterologous nucleic acid sequences such as, for example, regulatory sequences which direct in vivo expression of the receptor in a host cell.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleic acid molecules comprise a nucleotide sequence encoding a chimeric receptor comprising, from N-terminus to C-terminus: (a) an extracellular ligand binding domain having a binding affinity for a selected ligand; (b) a linking sequence having: (i) at least about 80% sequence identity to a polypeptide hinge domain; or (ii) at least about 80% sequence identity to a ROBO1 JMD including at least one fibronectin (Fn) repeat; (c) a TMD having at least about 80% sequence identity to the transmembrane domain of a Type 1 transmembrane receptor comprising one or more ligand-inducible proteolytic cleavage sites; and (d) an ICD comprising a transcriptional regulator, wherein binding of the selected ligand to the extracellular binding domain induces cleavage at the ligand-inducible proteolytic cleavage site between the transcriptional regulator and the linking sequence, and wherein the chimeric polypeptide does not comprise a sequence from a Notch receptor.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell or tissue and/or into an individual. Thus, in some embodiments, an expression cassette of the disclosure comprises a nucleotide sequence encoding a chimeric polypeptide operably linked to expression control elements sufficient to guide expression of the cassette in vivo. In some embodiments, the expression control element comprises a promoter and/or an enhancer and optionally, any or a combination of other nucleic acid sequences capable of effecting transcription and/or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. In some embodiments, a nucleic acid molecule encoding a chimeric receptor of the disclosure can be incorporated into one or more lentiviral vector constructs. In some embodiments, a nucleic acid molecule encoding a chimeric receptor of the disclosure can be incorporated into a single lentiviral vector construct. In some embodiments, a nucleic acid molecule encoding a chimeric receptor of the disclosure can be incorporated into two, three, four, five, or more lentiviral vector constructs.

In some embodiments, the recombinant nucleic acids encode a polypeptide with an amino acid sequence having at least about 80%, 90%, 95%, 96%, 97, 98%, 99%, or 100% sequence identity to a chimeric receptor disclosed herein. In some embodiments, the nucleic acid molecules encode a chimeric polypeptide comprising: (a) a linking sequence including an amino acid sequence having at least about 80%, 90%, 95%, 96%, 97, 98%, 99%, or 100% sequence identity to any one of SEQ ID NOS: 157-161; (b) a transmembrane domain including an amino acid sequence having at least about 80%, 90%, 95%, 96%, 97, 98%, 99%, or 100% sequence identity to any one of SEQ ID NOS: 1-84, 178, 182-185, and 190-192; and (c) an STS including an amino acid sequence having at least about 80%, 90%, 95%, 96%, 97, 98%, 99%, or 100% sequence identity to any one of SEQ ID NOS: 89, 92, 108, 115, 138, 146, and 156. In some embodiments, the nucleic acid molecules encode a chimeric polypeptide comprising: (a) a linking sequence including an amino acid sequence having at least about 90% sequence identity to any one of SEQ ID NO: 157-161; (b) a transmembrane domain including an amino acid sequence having at least about 90% sequence identity to any one of SEQ ID NOS: 1-84, 178, 182-185, and 190-192; and (c) an STS including an amino acid sequence having at least about 90% sequence identity to any one of SEQ ID NO: 89, 92, 108, 115, 138, 146, and 156. In some embodiments, the nucleic acid molecules encode a chimeric polypeptide comprising: (a) a linking sequence including an amino acid sequence having at least about 95% sequence identity to any one of SEQ ID NOS: 157-161; (b) a transmembrane domain including an amino acid sequence having at least about 95% sequence identity to any one of SEQ ID NOS: 1-84, 178, 182-185, and 190-192; and (c) an STS including an amino acid sequence having at least about 95% sequence identity to any one of SEQ ID NOS: 89, 92, 108, 115, 138, 146, and 156.

The nucleic acid sequences encoding the chimeric receptors can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, or bacteriophage, as a linear or circular, single-stranded or double-stranded, DNA or RNA polynucleotide, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005).Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). COR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors,

adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally but encode the same gene product because the genetic code is degenerate. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., comprising either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by the polymerase chain reaction (PCR). In the event the nucleic acid molecule is a ribonucleic acid (RNA), transcripts can be produced, for example, by in vitro transcription.

### Recombinant Cells and Cell Cultures

The nucleic acid of the present disclosure can be introduced into a host cell, such as a human T lymphocyte, to produce a recombinant cell containing the nucleic acid molecule. Accordingly, some embodiments of the disclosure relate to methods for making recombinant cells, including the steps of: (a) providing a cell capable of protein expression and (b) contacting the provided cell with any of the recombinant nucleic acids described herein.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules are delivered to cells by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. For example, introduction of nucleic acids into cells may be achieved by viral transduction. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also suitable for nucleic acid delivery and gene therapy via viral transduction. Lentiviral vectors offer several attractive properties as gene-delivery vehicles, including: (i) sustained gene delivery through stable vector integration into host genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells can be genetically engineered (e.g., transduced, transformed, or transfected) with, for example, a vector comprising a nucleic acid sequence encoding a chimeric receptor as described herein, either a virus-derived expression vector or a vector for homologous recombination further comprising nucleic acid sequences homologous to a portion of the genome of the host cell. Host cells can be either untransformed cells or cells that have already been transfected with one or more nucleic acid molecules.

In some embodiments, the recombinant cell is a prokaryotic cell or a eukaryotic cell. In some embodiments, the cell is transformed in vivo. In some embodiments, the cell is transformed ex vivo. In some embodiments, the cell is transformed in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a human subject.

In some embodiments, the recombinant cell further includes a second nucleic acid molecule as disclosed herein, wherein the first nucleic acid molecule and the second nucleic acid molecule do not have the same sequence. In some embodiments, the recombinant cell further includes a second chimeric polypeptide as disclosed herein, wherein the first chimeric polypeptide and the second chimeric polypeptide do not have the same sequence. In some embodiments, the first chimeric polypeptide modulates the expression and/or activity of the second chimeric polypeptide.

In some embodiments, the recombinant cell further includes an expression cassette encoding a protein of interest operably linked to a promoter, wherein expression of the protein of interest is modulated by the transcriptional regulator encoded by the chimeric receptor. In some embodiments, the protein of interest is heterologous to the recombinant cell. In principle, there are no particular limitations with regard to selecting proteins to target for modulation of expression by the transcriptional regulator encoded by the chimeric receptor. Non-limiting examples of proteins suitable for the regulation by the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporter genes. In some embodiments, the immune cell receptor comprises a T-cell receptor (TCR). A TCR generally comprises two polypeptides (e.g., polypeptide chains), such as an a-chain of a TCR, a β-chain of a TCR, a γ-chain of a TCR, a δ-chain of a TCR, or a combination thereof. Such polypeptide chains of TCRs are known in the art. In some embodiments, the immune cell receptor comprises a chimeric antigen receptor (CAR). Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., a carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the chimeric polypeptides and SynRIPRs of the disclosure can also be administered by transfection or infection using methods known in the art, including but not limited to the methods described in McCaffrey et al. (Nature (2002) 418:6893), Xia et al. (Nature Biotechnol (2002) 20:1006-10), or Putnam (Am J Health Syst Pharm (1996) 53:151-60, erratum at Am J Health Syst Pharm (1996) 53:325).

### Methods of the Disclosure

Administration of any one or more of the therapeutic compositions described herein, e.g., nucleic acids, recombinant cells, and pharmaceutical compositions, can be used to treat individuals having a disease, such as cancers and chronic infections. In some embodiments, the nucleic acids, recombinant cells, and pharmaceutical compositions are incorporated into therapeutic compositions for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, provided herein are methods for inhibiting an activity of a target cell in an individual, the methods comprising the step of administering to the individual a first therapy including one or more of the nucleic acids, recombinant cells, and pharmaceutical compositions provided herein, wherein the first therapy inhibits an activity of the target cell. For example, an activity of the target cell may be inhibited if its proliferation is reduced, if its pathologic or pathogenic behavior is reduced, if it is destroyed or killed, or the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits the target cell in the individual. Generally, the target cell of the disclosed methods can be any cell such as, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, provided herein are methods of treating a disease in an individual in need thereof, the methods comprising a step of administering to the individual a first therapy including one or more of chimeric polypeptides, SynRIPRs, nucleic acids, recombinant cells, or pharmaceutical compositions provided herein, wherein the first therapy treats the disease in the individual. In some embodiments, the methods include administering to the individual a first therapy including an effective number of the recombinant cells provided herein, wherein the recombinant cells treat the disease.

In another aspect, provided herein are methods for assisting in the treatment of a disease in an individual in need thereof, the methods comprising the steps of administering to the individual a first therapy comprising one or more recombinant nucleic acids, recombinant cells, or pharmaceutical compositions as disclosed herein, and administering to the individual a second therapy, wherein the first and second therapies together treat the disease in the individual. In some embodiments, the methods include administering to the individual a first therapy including an effective number of the recombinant cells as disclosed herein, wherein the recombinant cells treat the disease.

### Administration of Recombinant Cells to an Individual

In some embodiments, the methods involve administering an effective amount or number of the recombinant cells of the disclosure to an individual who is in need of such method. This administering step can be accomplished using any method of implantation known in the art. For example, the recombinant cells of the disclosure can be injected directly into the individual's bloodstream by intravenous infusion or otherwise administered to the individual.

The terms “administering”, “introducing”, and “transplanting” are used interchangeably herein to refer to methods of delivering recombinant cells expressing the chimeric receptors provided herein to an individual. In some embodiments, the methods comprise administering recombinant cells to an individual by a method or route of administration that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even long-term engraftment for the life time of the individual.

When provided prophylactically, in some embodiments, the recombinant cells described herein are administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source (i.e., the human subject being treated). In some embodiments, the recombinant cells are expanded in culture prior to administration to an individual in need thereof.

In some embodiments, the delivery of a composition comprising recombinant cells (i.e., a composition comprising a plurality of recombinant cells expressing any of the chimeric receptors provided herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment with a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting disease progression, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; (2) relieving the disease, e.g., causing regression of symptoms; or (3) preventing or reducing the likelihood of the development of symptoms.

In some embodiments, the individual is a mammal. In some embodiments, the mammal is human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. For example, the engineered CARs may be introduced into cells, e.g., T cells, using retroviruses, which efficiently and stably integrate a nucleic acid sequence encoding the chimeric antigen receptor into the target cell genome. An exemplary method is described in Examples 4 and 7 below.

Other methods known in the art include, but are not limited to, lentiviral transduction, transposon-based systems, direct RNA transfection, and CRISPR/Cas systems (e.g., type I, type II, or type III systems using a suitable Cas protein).

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh. 74. Production of pseudotyped rAAV is disclosed in, for example, international patent application publication number WO 01/83692.

The CAR-T cells, once they have been expanded ex vivo in response to, for example, an autoimmune disease antigen, can be reinfused into the subject in a therapeutically effective amount.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the CAR T cells of the present disclosure may be administered at a dosage of 10⁴ to 10¹⁰ total cells. In another embodiment, a pharmaceutical composition including the CAR T cells of the present disclosure may be administered at a dosage of 10³ to 10⁸ cells/kg body weight, including all integer values within those ranges.

Compositions including the CAR T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

### Additional Therapies

As discussed above, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Methods for Modulating an Activity of a Cell

In another aspect, provided herein are various methods for modulating an activity of a cell. The methods include: (a) providing a recombinant cell of the disclosure, and (b) contacting it with a selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. One skilled in the art upon reading the present disclosure will appreciate that the disclosed methods can be carried out in vivo, ex vivo, or in vitro.

Activities of a cell that can be modulated using a method of the present disclosure include, but are not limited to, expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

In some embodiments, the released transcriptional regulator modulates expression of a gene product of the cell. In some embodiments, the released transcriptional regulator modulates expression of a heterologous gene product in the cell. A heterologous gene product is one that is not normally produced by the cell. For example, the cell can be genetically modified with a nucleic acid comprising a nucleotide sequence encoding the heterologous gene product.

In some embodiments, the heterologous gene product is a secreted gene product. In some embodiments, the heterologous gene product is a cell surface gene product. In some cases, the heterologous gene product is a cytoplasmic gene product. In some embodiments, the released transcriptional regulator simultaneously modulates expression of two or more heterologous gene products in the cell.

In some embodiments, the heterologous gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor (TCR), a chimeric antigen receptor (CAR), a toxin, a toxin-derived protein, a transcriptional regulator, a transcriptional activator, a transcriptional repressor, a translation regulator, a translational activator, a translational repressor, an activating immuno-receptor, an antibody, an apoptosis inhibitor, an apoptosis inducer, an engineered T-cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.

The chimeric receptors of the disclosure provide a higher degree of expression than a standard SynNotch receptor, when using identical binding domains and ICDs. In some embodiments, depending on the ligand/binding domain pair and their affinity, the SynRIPR of the disclosure can provide expression enhancement of about 10%, about 15%, about 20%, about 25%, about 30%, about 35%, about 40%, about 45%, or about 50% higher than a corresponding SynNotch receptor.

Additionally, the chimeric receptors of the disclosure can provide transcriptional regulation that responds to the degree of T cell activation, independent of ligand binding. This permits additional flexibility in use, for example in cases where it is desired to enhance or suppress a T cell response when activated despite the absence of the chimeric receptor ligand.

### Systems and Kits

Also provided herein are systems and kits including the chimeric polypeptides, SynRIPRs, recombinant nucleic acids, recombinant cells, or pharmaceutical compositions provided and described herein as well as written instructions for making and using the same. For example, provided herein, in some embodiments, are systems and/or kits that include one or more of: a chimeric polypeptide as described herein, a SynRIPR as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided chimeric polypeptides, SynRIPRs, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g., CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Design and Construction of Chimeric Receptor and Response Element Constructs

This Example describes the design and construction of a family of non-Notch regulated intramembrane proteolysis receptors (SynRIPRs) receptors. Receptors were built by fusing the CD19 scFV (Porter et al., 2011) to an extracellular domain comprised of a truncated CD8α (P01732) hinge region (Thr138 to Cys164; SEQ ID NO: 158), a transmembrane domain (TMD) and intracellular juxtamembrane domain (STS as indicated below), and a transcriptional regulator. The transcriptional regulator GAL4-VP64 used in these experiments contained a DNA domain from yeast GAL4 transcription factor fused to an activation domain VP64, which consists of a tetrameric repeat of the minimal activation domain (amino acids 437-447) of the herpes simplex protein VP16.

Detailed information for various exemplary receptors of the disclosure can be found in Table 1 below.

TABLE 1
This table provides a brief description for each of the chimeric receptors of the disclosure, their
corresponding components, as well as corresponding sequence identifiers as set forth in the Sequence
Listing. ECD: extracellular domain; N-JMD: N-terminal juxtamembrane domain (i.e., hinge domain);
TMD: transmembrane domain; STS: stop-transfer-sequence; TF: transcriptional factor.

| | Receptor | | | | | | Full |
|---|---|---|---|---|---|---|---|
| Construct ID | Description | ECD | N-JMD | TMD | STS | TF | sequence |
| pIZ343GIVV | antiCD19scFv- | SEQ ID | SEQ ID | SEQ ID | SEQ ID | SEQ ID | SEQ ID |
| | CD8Hinge2- | NO: 174 | NO: 158 | NO: 176 | NO: 193 | NO: 196 | NO: 197 |
| | Notch1TMD_GIVVLLSmut- | | | | | | |
| | Notch1STS- | | | | | | |
| | Gal4VP64 | | | | | | |
| pIZ343GIVVa | antiCD19scFv- | SEQ ID | SEQ ID | SEQ ID | SEQ ID | SEQ ID | SEQ ID |
| | CD8Hinge2- | NO: 174 | NO: 158 | NO: 177 | NO: 193 | NO: 196 | NO: 198 |
| | Notch1TMD_GIVVSmut- | | | | | | |
| | Notch1STS- | | | | | | |
| | Gal4VP64 | | | | | | |
| pIZ373 | antiCD19scFv- | SEQ ID | SEQ ID | SEQ ID | SEQ ID | SEQ ID | SEQ ID |
| | IgG4Hinge(NoGSLinker)- | NO: 174 | NO: 175 | NO: 173 | NO: 193 | NO: 196 | NO: 199 |
| | Notch1TMD- | | | | | | |
| | Gal4VP64 | | | | | | |
| pIZ377 | antiCD19scFv- | SEQ ID | SEQ ID | SEQ ID | SEQ ID | SEQ ID | SEQ ID |
| | CD8Hinge2- | NO: 174 | NO: 158 | NO: 2 | NO: 108 | NO: 196 | NO: 200 |
| | CLSTN2TMD- | | | | | | |
| | PTPRFSTS- | | | | | | |
| | Gal4VP64 | | | | | | |
| pIZ378 | antiCD19scFv- | SEQ ID | SEQ ID | SEQ ID | SEQ ID | SEQ ID | SEQ ID |
| | CD8Hinge2- | NO: 174 | NO: 158 | NO: 2 | NO: 115 | NO: 196 | NO: 201 |
| | CLSTN2TMD- | | | | | | |
| | AGERSTS- | | | | | | |
| | Gal4VP64 | | | | | | |
| pIZ379 | antiCD19scFv- | SEQ ID | SEQ ID | SEQ ID | SEQ ID | SEQ ID | SEQ ID |
| | CD8Hinge2- | NO: 174 | NO: 158 | NO: 2 | NO: 146 | NO: 196 | NO: 202 |
| | CLSTN2TMD- | | | | | | |
| | KCNE3STS- | | | | | | |
| | Gal4VP64 | | | | | | |
| pTMD206 | antiCD19scFv- | SEQ ID | SEQ ID | SEQ ID | SEQ ID | SEQ ID | SEQ ID |
| | CD8Hinge2- | NO: 174 | NO: 158 | NO: 6 | NO: 193 | NO: 196 | NO: 203 |
| | APPTMD- | | | | | | |
| | Notch1STS- | | | | | | |
| | Gal4VP64 | | | | | | |
| pTMD206GV | antiCD19scFv- | SEQ ID | SEQ ID | SEQ ID | SEQ ID | SEQ ID | SEQ ID |
| | CD8Hinge2- | NO: 174 | NO: 158 | NO: 178 | NO: 193 | NO: 196 | NO: 204 |
| | APPTMD_GVmut- | | | | | | |
| | Notch1STS- | | | | | | |
| | Gal4VP64 | | | | | | |
| pTMD207 | antiCD19scFv- | SEQ ID | SEQ ID | SEQ ID | SEQ ID | SEQ ID | SEQ ID |
| | CD8Hinge2- | NO: 174 | NO: 158 | NO: 7 | NO: 193 | NO: 196 | NO: 205 |
| | BTCTMD- | | | | | | |
| | Notch1STS- | | | | | | |
| | Gal4VP64 | | | | | | |
| pTMD213 | antiCD19scFv- | SEQ ID | SEQ ID | SEQ ID | SEQ ID | SEQ ID | SEQ ID |
| | CD8Hinge2- | NO: 174 | NO: 158 | NO: 13 | NO: 193 | NO: 196 | NO: 206 |
| | CX3CL1TMD- | | | | | | |
| | Notch1STS- | | | | | | |
| | Gal4VP64 | | | | | | |
| pTMD214 | antiCD19scFv- | SEQ ID | SEQ ID | SEQ ID | SEQ ID | SEQ ID | SEQ ID |
| | CD8Hinge2- | NO: 174 | NO: 158 | NO: 14 | NO: 193 | NO: 196 | NO: 207 |
| | DCCTMD- | | | | | | |
| | Notch1STS- | | | | | | |
| | Gal4VP64 | | | | | | |
| pTMD215 | antiCD19scFv- | SEQ ID | SEQ ID | SEQ ID | SEQ ID | SEQ ID | SEQ ID |
| | CD8Hinge2- | NO: 174 | NO: 158 | NO: 15 | NO: 193 | NO: 196 | NO: 208 |
| | DLL1TMD- | | | | | | |
| | Notch1STS- | | | | | | |
| | Gal4VP64 | | | | | | |
| pTMD220 | antiCD19scFv- | SEQ ID | SEQ ID | SEQ ID | SEQ ID | SEQ ID | SEQ ID |
| | CD8Hinge2- | NO: 174 | NO: 158 | NO: 20 | NO: 193 | NO: 196 | NO: 209 |
| | EPCAMTMD- | | | | | | |
| | Notch1STS- | | | | | | |
| | Gal4VP64 | | | | | | |
| pTMD225 | antiCD19scFv- | SEQ ID | SEQ ID | SEQ ID | SEQ ID | SEQ ID | SEQ ID |
| | CD8Hinge2- | NO: 174 | NO: 158 | NO: 25 | NO: 193 | NO: 196 | NO: 210 |
| | ErbB4TMD- | | | | | | |
| | Notch1STS- | | | | | | |
| | Gal4VP64 | | | | | | |
| pTMD244 | antiCD19scFv- | SEQ ID | SEQ ID | SEQ ID | SEQ ID | SEQ ID | SEQ ID |
| | CD8Hinge2- | NO: 174 | NO: 158 | NO: 44 | NO: 193 | NO: 196 | NO: 211 |
| | LRP1TMD- | | | | | | |
| | Notch1STS- | | | | | | |
| | Gal4VP64 | | | | | | |
| pTMD247 | antiCD19scFv- | SEQ ID | SEQ ID | SEQ ID | SEQ ID | SEQ ID | SEQ ID |
| | CD8Hinge2- | NO: 174 | NO: 158 | NO: 47 | NO: 193 | NO: 196 | NO: 212 |
| | LRP6TMD- | | | | | | |
| | Notch1STS- | | | | | | |
| | Gal4VP64 | | | | | | |
| pTMD253 | antiCD19scFv- | SEQ ID | SEQ ID | SEQ ID | SEQ ID | SEQ ID | SEQ ID |
| | CD8Hinge2- | NO: 174 | NO: 158 | NO: 53 | NO: 193 | NO: 196 | NO: 213 |
| | SCN4BTMD- | | | | | | |
| | Notch1STS- | | | | | | |
| | Gal4VP64 | | | | | | |
| pTMD254 | antiCD19scFv- | SEQ ID | SEQ ID | SEQ ID | SEQ ID | SEQ ID | SEQ ID |
| | CD8Hinge2- | NO: 174 | NO: 158 | NO: 54 | NO: 193 | NO: 196 | NO: 214 |
| | NECTIN1TMD- | | | | | | |
| | Notch1STS- | | | | | | |
| | Gal4VP64 | | | | | | |
| pTMD258 | antiCD19scFv- | SEQ ID | SEQ ID | SEQ ID | SEQ ID | SEQ ID | SEQ ID |
| | CD8Hinge2- | NO: 174 | NO: 158 | NO: 179 | NO: 193 | NO: 196 | NO: 215 |
| | Notch2TMD- | | | | | | |
| | Notch1STS- | | | | | | |
| | Gal4VP64 | | | | | | |
| pTMD259 | antiCD19scFv- | SEQ ID | SEQ ID | SEQ ID | SEQ ID | SEQ ID | SEQ ID |
| | CD8Hinge2- | NO: 174 | NO: 158 | NO: 180 | NO: 193 | NO: 196 | NO: 216 |
| | Notch3TMD- | | | | | | |
| | Notch1STS- | | | | | | |
| | Gal4VP64 | | | | | | |
| pTMD260 | antiCD19scFv- | SEQ ID | SEQ ID | SEQ ID | SEQ ID | SEQ ID | SEQ ID |
| | CD8Hinge2- | NO: 174 | NO: 158 | NO: 181 | NO: 193 | NO: 196 | NO: 217 |
| | Notch4TMD- | | | | | | |
| | Notch1STS- | | | | | | |
| | Gal4VP64 | | | | | | |
| pTMD260GV | antiCD19scFv- | SEQ ID | SEQ ID | SEQ ID | SEQ ID | SEQ ID | SEQ ID |
| | CD8Hinge2- | NO: 174 | NO: 158 | NO: 181 | NO: 193 | NO: 196 | NO: 218 |
| | Notch4TMDGVmut- | | | | | | |
| | Notch1STS- | | | | | | |
| | Gal4VP64 | | | | | | |
| pTMD268 | antiCD19scFv- | SEQ ID | SEQ ID | SEQ ID | SEQ ID | SEQ ID | SEQ ID |
| | CD8Hinge2- | NO: 174 | NO: 158 | NO: 64 | NO: 193 | NO: 196 | NO: 219 |
| | PCDHGC3TMD- | | | | | | |
| | Notch1STS- | | | | | | |
| | Gal4VP64 | | | | | | |
| pTMD270 | antiCD19scFv- | SEQ ID | SEQ ID | SEQ ID | SEQ ID | SEQ ID | SEQ ID |
| | CD8Hinge2- | NO: 174 | NO: 158 | NO: 66 | NO: 193 | NO: 196 | NO: 220 |
| | AGERTMD- | | | | | | |
| | Notch1STS- | | | | | | |
| | Gal4VP64 | | | | | | |
| pTMD270GVLVS | antiCD19scFv- | SEQ ID | SEQ ID | SEQ ID | SEQ ID | SEQ ID | SEQ ID |
| | CD8Hinge2- | NO: 174 | NO: 158 | NO: 182 | NO: 193 | NO: 196 | NO: 221 |
| | AGERTMD_GVLVSmut- | | | | | | |
| | Notch1STS- | | | | | | |
| | Gal4VP64 | | | | | | |
| pTMD270LL | antiCD19scFv- | SEQ ID | SEQ ID | SEQ ID | SEQ ID | SEQ ID | SEQ ID |
| | CD8Hinge2- | NO: 174 | NO: 158 | NO: 183 | NO: 193 | NO: 196 | NO: 222 |
| | AGERTMD_LLmut- | | | | | | |
| | Notch1STS- | | | | | | |
| | Gal4VP64 | | | | | | |
| pTMD272 | antiCD19scFv- | SEQ ID | SEQ ID | SEQ ID | SEQ ID | SEQ ID | SEQ ID |
| | CD8Hinge2- | NO: 174 | NO: 158 | NO: 68 | NO: 193 | NO: 196 | NO: 223 |
| | PTPRMTMD- | | | | | | |
| | Notch1STS- | | | | | | |
| | Gal4VP64 | | | | | | |
| pTMD273 | antiCD19scFv- | SEQ ID | SEQ ID | SEQ ID | SEQ ID | SEQ ID | SEQ ID |
| | CD8Hinge2- | NO: 174 | NO: 158 | NO: 69 | NO: 193 | NO: 196 | NO: 224 |
| | ROBO1TMD- | | | | | | |
| | Notch1STS- | | | | | | |
| | Gal4VP64 | | | | | | |
| pTMD273GV | antiCD19scFv- | SEQ ID | SEQ ID | SEQ ID | SEQ ID | SEQ ID | SEQ ID |
| | CD8Hinge2- | NO: 174 | NO: 158 | NO: 69 | NO: 193 | NO: 196 | NO: 225 |
| | ROBO1TMD_GVmut- | | | | | | |
| | Notch1STS- | | | | | | |
| | Gal4VP64 | | | | | | |
| pTMD273GVLLS | antiCD19scFv- | SEQ ID | SEQ ID | SEQ ID | SEQ ID | SEQ ID | SEQ ID |
| | CD8Hinge2- | NO: 174 | NO: 158 | NO: 184 | NO: 193 | NO: 196 | NO: 226 |
| | ROBO1TMD_GVLVSmut- | | | | | | |
| | Notch1STS- | | | | | | |
| | Gal4VP64 | | | | | | |
| pTMD276 | antiCD19scFv- | SEQ ID | SEQ ID | SEQ ID | SEQ ID | SEQ ID | SEQ ID |
| | CD8Hinge2- | NO: 174 | NO: 158 | NO: 72 | NO: 193 | NO: 196 | NO: 227 |
| | SORL1TMD- | | | | | | |
| | Notch1STS- | | | | | | |
| | Gal4VP64 | | | | | | |
| pTMD277 | antiCD 19scFv- | SEQ ID | SEQ ID | SEQ ID | SEQ ID | SEQ ID | SEQ ID |
| | CD8Hinge2- | NO: 174 | NO: 158 | NO: 73 | NO: 193 | NO: 196 | NO: 228 |
| | SORT1TMD- | | | | | | |
| | Notch1STS- | | | | | | |
| | Gal4VP64 | | | | | | |
| pTMD278 | antiCD19scFv- | SEQ ID | SEQ ID | SEQ ID | SEQ ID | SEQ ID | SEQ ID |
| | CD8Hinge2- | NO: 174 | NO: 158 | NO: 74 | NO: 193 | NO: 196 | NO: 229 |
| | SDC1TMD- | | | | | | |
| | Notch1STS- | | | | | | |
| | Gal4VP64 | | | | | | |
| pTMD279 | antiCD19scFv- | SEQ ID | SEQ ID | SEQ ID | SEQ ID | SEQ ID | SEQ ID |
| | CD8Hinge2- | NO: 174 | NO: 158 | NO: 75 | NO: 193 | NO: 196 | NO: 230 |
| | SDC2TMD- | | | | | | |
| | Notch1STS- | | | | | | |
| | Gal4VP64 | | | | | | |
| pTMD280 | antiCD19scFv- | SEQ ID | SEQ ID | SEQ ID | SEQ ID | SEQ ID | SEQ ID |
| | CD8Hinge2- | NO: 174 | NO: 158 | NO: 76 | NO: 193 | NO: 196 | NO: 231 |
| | SDC3TMD- | | | | | | |
| | Notch1STS- | | | | | | |
| | Gal4VP64 | | | | | | |
| pTMD289 | antiCD19scFv- | SEQ ID | SEQ ID | SEQ ID | SEQ ID | SEQ ID | SEQ ID |
| | CD8Hinge2- | NO: 174 | NO: 158 | NO: 185 | NO: 193 | NO: 196 | NO: 232 |
| | Notchl_DrerioTMD- | | | | | | |
| | Notch1STS- | | | | | | |
| | Gal4VP64 | | | | | | |
| pTMD290 | antiCD19scFv- | SEQ ID | SEQ ID | SEQ ID | SEQ ID | SEQ ID | SEQ ID |
| | CD8Hinge2- | NO: 174 | NO: 158 | NO: 186 | NO: 193 | NO: 196 | NO: 233 |
| | Notch1_DmelanogasterTMD- | | | | | | |
| | Notch1STS- | | | | | | |
| | Gal4VP64 | | | | | | |
| pTMD291 | antiCD19scFv- | SEQ ID | SEQ ID | SEQ ID | SEQ ID | SEQ ID | SEQ ID |
| | CD8Hinge2- | NO: 174 | NO: 158 | NO: 187 | NO: 193 | NO: 196 | NO: 234 |
| | Notch1_XlaevisTMD- | | | | | | |
| | Notch1STS- | | | | | | |
| | Gal4VP64 | | | | | | |
| pTMD292 | antiCD19scFv- | SEQ ID | SEQ ID | SEQ ID | SEQ ID | SEQ ID | SEQ ID |
| | CD8Hinge2- | NO: 174 | NO: 158 | NO: 188 | NO: 193 | NO: 196 | NO: 235 |
| | Notch1_GgallusTMD- | | | | | | |
| | Notch1STS- | | | | | | |
| | Gal4VP64 | | | | | | |
| pTMD295 | antiCD19scFv- | SEQ ID | SEQ ID | SEQ ID | SEQ ID | SEQ ID | SEQ ID |
| | CD8Hinge2- | NO: 174 | NO: 158 | NO: 189 | NO: 193 | NO: 196 | NO: 236 |
| | Notch1_MmusculusTMD- | | | | | | |
| | Notch1STS- | | | | | | |
| | Gal4VP64 | | | | | | |
| pTMD297pre | antiCD19scFv- | SEQ ID | SEQ ID | SEQ ID | SEQ ID | SEQ ID | SEQ ID |
| | CD8Hinge- | NO: 174 | NO: 157 | NO: 190 | NO: 193 | NO: 196 | NO: 237 |
| | CD8aTMD- | | | | | | |
| | Notch1STS- | | | | | | |
| | Gal4VP64 | | | | | | |
| pTMD297pre_GV | antiCD19scFv- | SEQ ID | SEQ ID | SEQ ID | SEQ ID | SEQ ID | SEQ ID |
| | CD8Hinge- | NO: 174 | NO: 157 | NO: 191 | NO: 193 | NO: 196 | NO: 238 |
| | CD8aTMDGV- | | | | | | |
| | Notch1STS- | | | | | | |
| | Gal4VP64 | | | | | | |
| pTMD297pre_GVLVS | antiCD19scFv- | SEQ ID | SEQ ID | SEQ ID | SEQ ID | SEQ ID | SEQ ID |
| | CD8Hinge- | NO: 174 | NO: 157 | NO: 192 | NO: 193 | NO: 196 | NO: 239 |
| | CD8aTMD_GVLVSmut- | | | | | | |
| | Notch1STS- | | | | | | |
| | Gal4VP64 | | | | | | |
| pSTS205 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 89 | NO: 196 | NO: 240 |
| | Notch1TMD- | | | | | | |
| | CSFIRSTS- | | | | | | |
| | Gal4VP64 | | | | | | |
| pSTS231 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 115 | NO: 196 | NO: 241 |
| | Notch1TMD- | | | | | | |
| | AGERSTS- | | | | | | |
| | Gal4VP64 | | | | | | |
| pSTS262 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 146 | NO: 196 | NO: 242 |
| | Notch1TMD- | | | | | | |
| | KCNE3STS- | | | | | | |
| | Gal4VP64 | | | | | | |
| pSTS263 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 147 | NO: 196 | NO: 243 |
| | Notch1TMD- | | | | | | |
| | CDH2STS- | | | | | | |
| | Gal4VP64 | | | | | | |
| pSTS266 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 150 | NO: 196 | NO: 244 |
| | Notch1TMD- | | | | | | |
| | BTCSTS- | | | | | | |
| | Gal4VP64 | | | | | | |
| pSTS267 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 151 | NO: 196 | NO: 245 |
| | Notch1TMD- | | | | | | |
| | EPHA4STS- | | | | | | |
| | Gal4VP64 | | | | | | |
| pSTS268 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 101 | NO: 196 | NO: 246 |
| | Notch1TMD- | | | | | | |
| | ILIR2STS- | | | | | | |
| | Gal4VP64 | | | | | | |
| pSTS274 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 194 | NO: 196 | NO: 247 |
| | Notch1TMD- | | | | | | |
| | Notch3STS- | | | | | | |
| | Gal4VP64 | | | | | | |
| pIZ364 | antiCD19scFv- | SEQ ID | SEQ ID | SEQ ID | SEQ ID | SEQ ID | SEQ ID |
| | CD8Hinge2- | NO: 174 | NO: 158 | NO: 173 | NO: 195 | NO: 196 | NO: 248 |
| | Notch1TMD- | | | | | | |
| | Notch4STS- | | | | | | |
| | Gal4VP64 | | | | | | |

All receptors contain an N-terminal CD8α signal peptide (MALPVTALLLPLALLLHAARP; SEQ ID NO: 162) for membrane targeting and a myc-tag (EQKLISEEDL; SEQ ID NO: 163) for suitable determination of surface expression with a-myc AF647 (Cell-Signaling #2233). For the construction of these receptors, DNA fragments coding for the amino acid sequences provided in the Sequence Listing were PCR amplified from synthesized gene fragments or plasmids containing DNA sequence for the indicated protein, and assembled using standard cloning techniques (e.g., overhang PCR, fusion PCR, and In-fusion cloning) with flanking translation start and stop sequences, into a modified pHR′SIN:CSW vector (K.T. Roybal et al., Cell (2016) 167(2):419-32) containing a 3-phosphoglycerate kinase (PGK) promoter for all primary T cell experiments described below.

The pHR′SIN:CSW vector was also modified to produce the response element plasmids. For this purpose, five copies of a target sequence for binding of GAL4 DBD domain (GGAGCACTGTCCTCCGAACG; SEQ ID NO: 164) were cloned 5′ to a minimal synthetic pybTATA promoter.

Also included in the response element plasmids is a PGK promoter that either constitutively drives expression of a fluorophore (mCitrine or mCherry) to suitably identify transduced T cells or a SynRIPR for single vector experimentation. For all inducible chimeric antigen-receptor (CAR) vectors, the CARs were tagged N-terminally with Flag-tag and were co-induced with GFP using a T2A element. All induced elements were cloned via a BamHI site in the multiple cloning located 3′ to the Gal4 response elements. All constructs were cloned via In-Fusion® cloning (Takara # 638951) according to the manufacturer's instructions.

### Example 2

### Primary Human T-Cell Isolation and Culture

This Example describes the isolation and culture of primary human T cells that were subsequently used in various cell transduction experiments described in Example 3 below.

In these experiments, primary CD4⁺ and CD8⁺ T cells were isolated from anonymous donor blood after apheresis and enriched by negative selection using human T-cell isolation kits (human CD4⁺ or CD8⁺ enrichment cocktail; STEMCELL Technologies Cat #15062 and 15063). Blood was obtained from Blood Centers of the Pacific (San Francisco, CA) as approved by the University Institutional Review Board. T cells were cryopreserved in growth medium (RPMI-1640, UCSF cell culture core) with 20% human AB serum (Valley Biomedical Inc., #HP1022) and 10% DMSO. After thawing, T cells were cultured in human T cell medium containing X-VIVO™ 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Example 3

### Human T Cells Were Stably Transduced with Lentiviral Vectors

The Example describes a general protocol used for lentiviral transduction of human T cells.

Generally, lentiviral vectors pseudo-typed with vesicular stomatitis virus envelope G protein (VSV-G) (pantropic vectors) were produced via transfection of Lenti-X™ 293T cells (Clontech #11131D) with a pHR'SIN:CSW transgene expression vector and the viral packaging plasmids pCMVdR8.91 and pMD2.G using Mirus TransIT®-Lenti (Mirus, #MIR 6606). Generally, primary T cells were thawed the same day and, after 24 hours in culture, were stimulated with beads having anti-CD3 and anti-CD28 antibodies bound to the surface (Human T-Activator CD3/CD28 Dynabeads®, Life Technologies #11131D) at a 1:3 cell:bead ratio. At 48 hours, viral supernatant was harvested and the primary T cells were exposed to the virus for 24 hours. At Day 5 post T-cell stimulation, the beads were removed, and the T cells expanded until Day 14 when they were rested and could be used in assays. T cells were sorted for assays with a Beckton Dickinson (BD Biosciences) FACSAria™ II flow cytometer. Sorted T-cells were expanded until Day 10 for in vivo assays and until Day 14 for in vitro assays.

### Example 4

### In Vitro SynRIPR Activation Assays

For all in vitro SynRIPR activation assays, 1×10⁵ T cells or Jurkat T cells were co-cultured with target cells at a 1:1 ratio in 96 well round bottom plates (VWR). To exogenously activate T-cells, 100 ng/mL PMA (Sigma #P1575) or MCAM BiTEs were added to co-cultures. When activating a co-expressed ALPPL2 CAR, ALPPL2+ K562 cells were added to the co-culture in a 1:1 ratio with T cells. The cultures were analyzed at the time points indicated for reporter activation using a BD FACSymphony Fortessa X-50. All flow cytometry analysis was performed in FlowJo software (BD).

### Example 5

### Cancer Cell Lines

This Example describes the generation of myelogenous leukemia “sender” cells expressing CD19 at equivalent levels as Daudi tumors.

The cancer cell lines used were K562 myelogenous leukemia cells (ATCC #CCL-243) and Daudi B cell lymphoblasts (ATCC #CCL-213). The K562 cells were lentivirally transduced to stably express human CD19 at equivalent levels as Daudi tumors. CD19 levels were determined by staining the cells with α-CD19 APC (Biolegend® #302212). All cell lines were sorted for expression of the transgenes.

### Example 6

### Generation of Reporter Jurkat T Cells For Screening

This Example describes the generation of reporter Jurkat T cells that were subsequent used for the screening of various synthetic receptors containing non-Notch transmembrane domains (TMD) and/or stop-transfer sequences (STS).

In these experiments, E6-1 Jurkat T cells (ATCC# TIB-152) were lentivirally transduced with a reporter plasmid carrying an inducible Gal4-driven BFP reporter gene and a constitutive expressed mCitrine reporter gene, as described previously (K.T. Roybal et al., Cell, 164:1-10, 2016). Reporter-positive Jurkat cells were sorted for mCitrine positive expression using a Beckton Dickinson (BD Biosciences) FACSAria™ II flow cytometer and expanded.

Lentiviral particles were produced with the receptor transgene expression vector as described previously (L. Morsut et al., Cell (2016) 164:780-91). Individual cultures of reporter-positive Jurkat T cells were lentivirally transduced in a 96 well plate with myc-tagged α-CD19 human SynNotch1 receptors with modified transmembrane domain (TMD) or juxtamembrane domains (STS). After viral transduction, the receptor transduction efficiency for each Jurkat cell population was measured with a BD FACSymphony Fortessa X-50 following staining with anti-myc AF647 (Cell-Signaling #2233).

### Example 7

### Non-Notch Synthetic Regulated Intramembrane Proteolysis Receptors (SynRIPRs)

This Example describes experiments performed to demonstrate the regulation of intramembrane proteolysis in vitro by exemplary chimeric non-Notch polypeptides described herein.

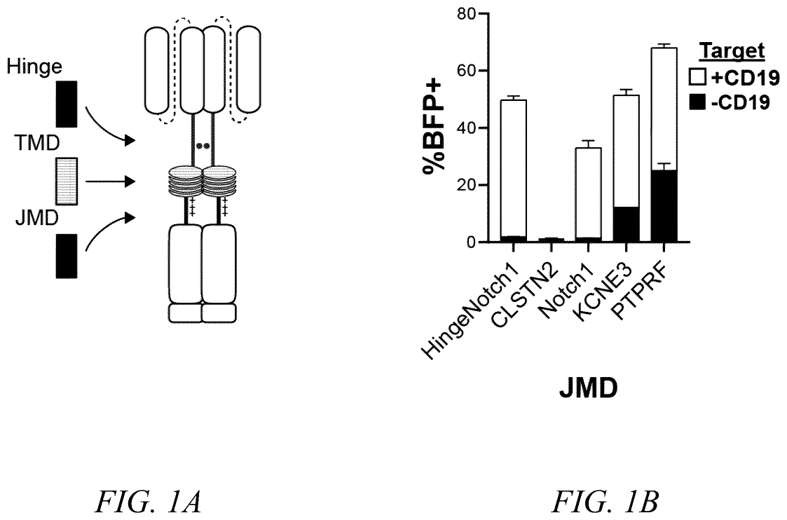

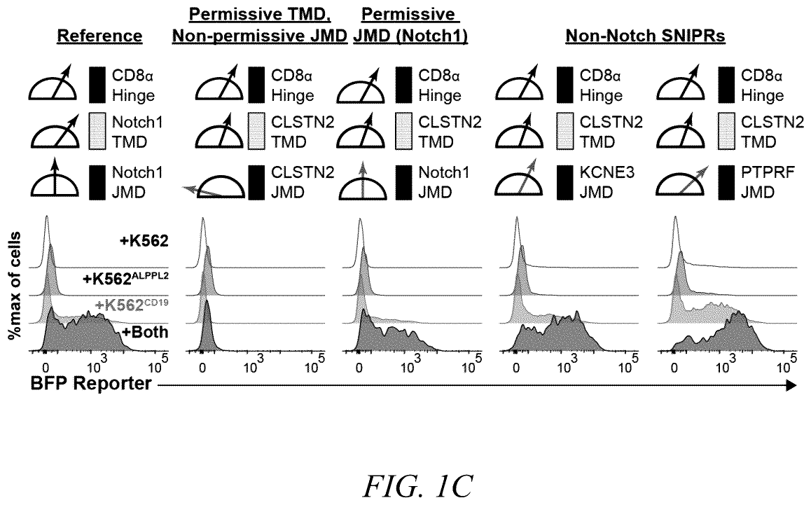

In these experiments, the reference receptor was an optimized CD8α Notch receptor (HingeNotch) composed of a truncated CD8α extracellular domain (ECD), a transmembrane domain (TMD) of human Notch1, and a C-terminal juxtamembrane domain (JMD/STS) from Notch1. T cells expressing this anti-CD19 HingeNotch were able to activate a response element in the presence of CD19+ K562 sender cells. This activation was boosted when a co-expressed anti-ALPPL2 CAR is activated, providing a T cell activation stimulus. The results of these experiments are schematically presented in FIGS. 1A-1C.

It was observed that although all receptors expressed at high levels, the otherwise-active Alcadein-gamma (CSTLN2) TMD did not function with its cognate, but inactive, STS/JMD sequence. In particular, by replacing the Notch1 TMD and JMD/STS with the equivalent domains from CLSTN2, it was observed that receptor activity was undetectable. Although the CLSTN2 TMD performed well in prior TMD screening experiments (data not shown), the CLSTN2 JMD/STS was found to be non-functional. It was also observed that by restoring the Notch1 JMD, the receptor activity with the CLSTN2 TMD was restored.

Remarkably, it was observed that receptor signaling was restored by replacing the CSTLN2 JMD/STS with a more active JMD/STS from KCNE3 (RSRKVDKR; SEQ ID NO: 146) or PTPRF (KRKRTH; SEQ ID NO: 108), e.g., producing a functional receptor when paired with the CLSTN2 TMD. This result indicates that the original CLSTN2 JMD was functionally limiting.

In summary, the experimental results described in this Example demonstrate that assembly of a non-Notch SynRIPR using the CLSTN2 TMD and JMD results in an inactive receptor, however receptor function was restored when the CLSTN2 JMD was replaced with a higher performing JMD.

As discussed above, all existing functional SynNotch receptors have included sequence derived from Notch family members. To demonstrate the versatility of the modular receptor assembly approach, the experiments described in this Example have been designed to engineer ligand-activated receptors from the ground up based on receptor components that were completely devoid of Notch sequences. The experimental results described herein illustrate the ability to build new functional synthetic regulated intramembrane proteolysis receptors from a set of functional parts, which in turns demonstrates that synthetic biology approach of receptor assembly is capable of generating receptors that function like Notch receptors but can be rationally customized in their activity.

### Example 8

### Modular Assembly of SynRIPRs

This Example describes experiments performed to illustrate that synthetic receptors can be assembled in a modular fashion by combining a ligand binding domain, an ECD, a TMD, a JMD, and a transcription factor.

In these experiments, an optimized CD8α Hinge ECD was selected for additional testing due to its robust expression and lack of ligand-independent signaling. In the experiments below, the Notch1 JMD (STS) was kept constant for TMD selection, whereas the Notch1 TMD was kept constant for JMD (STS) selection.

For selection of TMD, this CD8α Hinge ECD was combined with the anti-CD19 scFV, a variety of high-performing TMDs from previous screening, the Notch1 JMD, and transcriptional regulator Gal4VP64. T cells expressing these receptors were co-incubated with CD19+ sender K562 cells for 48 hours and production of a BFP reporter was measured using flow cytometry. The Notch1 TMD and two TMD mutants were selected due to their low levels of ligand-independent signaling.

For selection of JMD, the optimized CD8α Hinge ECD was paired with the anti-CD19 scFV, the Notch1 TMD, a variety of JMDs, and transcriptional regulator Gal4VP64. T cells expressing these receptors were co-incubated with CD19+ sender K562 cells and production of a BFP reporter was measured using flow cytometry. The Notch1, NRG1, and Notch2 JMDs were selected due to their variable output production levels allowing for signal tuning.

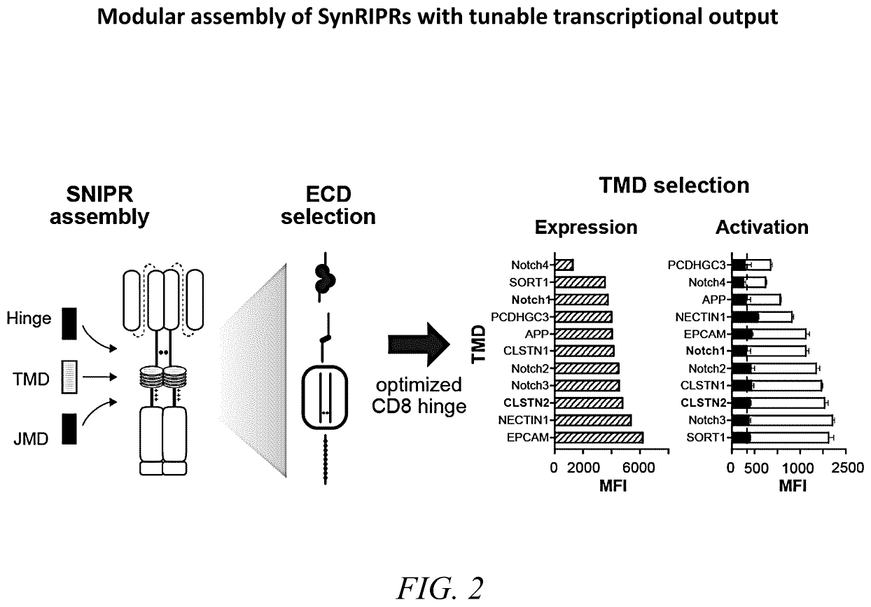

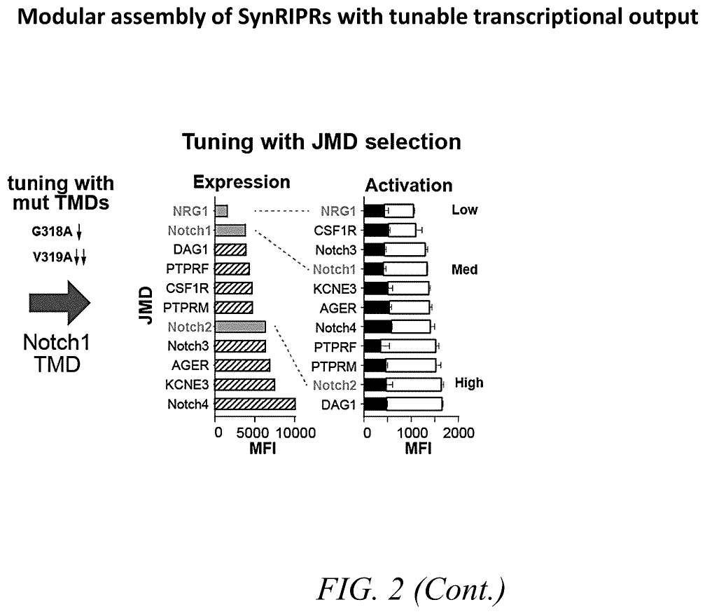

In these experiments, T cells expressing high-performing “SynRIPR→BFP expression” circuits were co-incubated with sender cells for 48 hours. BFP output was measured using flow cytometry. The results of these experiments are schematically presented in FIG. 2.

In summary, the experiments described herein illustrate that synthetic regulated intramembrane proteolysis receptor scaffolds suitable for clinical translation can be designed and assembled by choosing an ECD, TMD, and JMD for further testing. Exemplary design criteria included robust expression, high ligand-dependent activation, and low ligand-independent activation under both basal and activated T cell conditions. In one exemplary workflow, one could initially select the optimized CD8α-hinge ECD, due to its strong expression and selective response to ligand, and screened it with a selection of high-performing TMDs and JMDs. As the assembly of SynRIPRs suggests a roughly additive effect for each component to total receptor function, an optimization search could be simplified by screening through TMDs and JMDs with constant Notch1 complementary components. From experimentation, a decision was made to maintain the Notch1 TMD due to its robust activation and best-in-class levels of ligand-independent signaling, along with the ready availability of mutants for tunability. This ECD-TMD combination was screened against a panel of JMDs, choosing a set of SNIPRs (synthetic intramembrane proteolysis receptors) with a range of activation levels. It was observed that although SynRIPR expression levels varied between TMDs and JMDs, these differences did not entirely correlate with receptor activation, which seems to indicate a more pleotropic role for the JMD.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

David L. Porter, M.D., Bruce L. Levine, Ph.D., Michael Kalos, Ph.D., Adam Bagg, M.D., and Carl H. June, M.D. Chimeric Antigen Receptor-Modified T Cells in Chronic Lymphoid Leukemia. N. Engl J Med. (2011) Aug 25; 365(8): 725-33.
Gordon WR et al., The molecular logic of Notch signaling—a structural and biochemical perspective. J. Cell Sci. (2008) 121:3109-19.
Gordon WR et al., Mechanical Allostery: Evidence for a Force Requirement in the Proteolytic Activation of Notch. Dev Cell (2015) 33:729-36.
Haapasalo Al, Kovacs DM. The many substrates of presenilin/γ-secretase. J Alzheimers Dis. (2011);25(1):3-28.
Rosmalen M., Krom M., and Merkx M. Tuning the Flexibility of Glycine-Serine Linkers To Allow Rational. Biochemistry (2017) 56:6565-6574.
Morsut L, Roybal KT, Xiong X, Gordley RM, Coyle SM, Thomson M, and Lim WA. Engineering Customized Cell Sensing and Response Behaviors Using Synthetic Notch Receptors. Cell. (2016) February 11; 164(4):780-91.
Naso MF, Tomkowicz B, Perry WL 3rd, Strohl WR. Adeno-Associated Virus (AAV) as a Vector for Gene Therapy. BioDrugs. (2017); 31(4):317-34.
Nasri M, Karimi A, Allahbakhshian Farsani M. Production, purification and titration of a lentivirus-based vector for gene delivery purposes. Cytotechnology. (2014); 66(6):1031-38.
Roybal KT, Jasper Z. Williams, Leonardo Morsut, Levi J. Rupp, Isabel Kolinko, Joseph H. Choe, Whitney J. Walker, Krista A. McNally, and Wendell A. Lim. Engineering T cells with Customized Therapeutic Response Programs Using Synthetic Notch Receptors. Cell. (2016) Oct 6; 167(2):419-32.
Samulski and Muzyczka. AAV-Mediated Gene Therapy for Research and Therapeutic Purposes. Annu. Rev. Virol. (2014) 1:427.
Sakuma, et al. (2012). Lentiviral vectors: basic to translational. Biochem. J. 443:603.
Watson D.J., Wolfe J.H. Viral vectors for gene therapy: methods and protocols. Totowa, NJ, USA: Humana Press; (2003). pp. 383-404.
Vidarsson G. et al., IgG subclasses and allotypes: from structure to effector functions. Frontiers Immunol. (2014) Oct 20; 5:520.
Zhang et al., The γ-secretase complex: from structure to function. Frontiers in Cellular Neuroscience. (2014) December 8: 427.

What is claimed is:

1. A chimeric polypeptide comprising, from N-terminus to C-terminus:
a) an extracellular binding domain (ECD) having a binding affinity for a selected ligand;
b) a linking sequence;
c) a transmembrane domain(TMD) having at least about 80% sequence identity to the transmembrane domain of a Type 1 transmembrane receptor and comprising one or more ligand-inducible proteolytic cleavage sites; and
d) an intracellular domain (ICD) comprising a transcriptional regulator, wherein binding of the selected ligand to the extracellular binding domain induces cleavage at the ligand-inducible proteolytic cleavage site between the transcriptional regulator and the linking sequence,
and wherein the chimeric polypeptide does not comprise a sequence from a Notch receptor.

2. The chimeric polypeptide of claim 1, further comprising a stop-transfer sequence (STS) positioned between the TMD and the ICD, wherein the STS is heterologous to the TMD.

3. The chimeric polypeptide of any one of claims 1 to 2, wherein the TMD comprises a polypeptide sequence having at least 80% sequence identity to a transmembrane domain from a Type 1 transmembrane receptor and comprises a γ-secretase cleavage site.

4. The chimeric polypeptide of any one of claims 1 to 3, wherein the TMD comprises a polypeptide sequence having at least 90% sequence identity to a transmembrane domain from a Type 1 transmembrane receptor and comprises a γ-secretase cleavage site.

5. The chimeric polypeptide of any one of claims 1 to 4, wherein the TMD comprises a polypeptide sequence having at least 95% sequence identity to a transmembrane domain from a Type 1 transmembrane receptor and comprises a γ-secretase cleavage site.

6. The chimeric polypeptide of any one of claims 1 to 5, wherein the Type 1 transmembrane receptor is selected from the group consisting of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, IFNAR2, ILIR1, ILIR2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SORT1, SDC1, SDC2, TYR, TYRP1, DCT, VASN, FLT1, CDH5, PKHD1, NECTIN1, PCDHGC3, NRG1, LRPIB, CDH2, NRG2, PTPRK, SCN2B, Nradd, and PTPRM, and comprises a γ-secretase cleavage site.

7. The chimeric polypeptide of claim 6, wherein the Type 1 transmembrane receptor is APP, CLSTN1, CLSTN2, EPCAM, KCNE3, NECTIN1, PCDHGC3, and PTPRF.

8. The chimeric polypeptide of any one of claims 1 to 7, wherein the STS comprises a sequence having at least 70% sequence identity to a STS sequence from CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, PTPRF, or PTPRM.

9. The chimeric polypeptide of claim 8, wherein the STS comprises a sequence having at least 70% sequence identity to a STS sequence from CSF1R, DAG1, AGER, NRG1, KCNE3, PTPRF, or PTPRM.

10. The chimeric polypeptide of any one of claims 1 to 9, wherein the extracellular domain comprises an antigen-binding moiety capable of binding to a ligand on the surface of a cell.

11. The chimeric polypeptide of any one of claims 1 to 10, wherein the cell is a pathogenic cell.

12. The chimeric polypeptide of any one of claims 1 to 11, wherein the ligand comprises a protein or a carbohydrate.

13. The chimeric polypeptide of any one of claims 1 to 12, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE, Alkaline phosphatase, placental-like 2 (ALPPL2), B-cell maturation antigen (BCMA), Green Fluorescent Protein (GFP), Enhanced Green Fluorescent Protein (EGFP), and Signal regulatory protein a (SIRPα).

14. The chimeric polypeptide of any one of claims 1 to 13, wherein the ligand is selected from cell surface receptors, adhesion proteins, integrins, mucins, lectins, tumor-associated antigens, and tumor-specific antigens.

15. The chimeric polypeptide of any one of claims 1 to 14 wherein the ligand is a tumor-associated antigen or a tumor-specific associated antigen.

16. The chimeric polypeptide of any one of claims 1 to 15, wherein the extracellular binding domain comprises the ligand-binding portion of a receptor.

17. The chimeric polypeptide of any one of claims 10 to 16, wherein the antigen-binding moiety is selected from the group consisting of an antibody, a nanobody, a diabody, a triabody, or a minibody, a F(ab′)₂ fragment, a Fab fragment, a single chain variable fragment (scFv), a single domain antibody (sdAb), or a functional fragment thereof.

18. The chimeric polypeptide of claim 17, wherein the antigen-binding moiety comprises an scFv.

19. The chimeric polypeptide of any one of claims 10 to 18, wherein the antigen-binding moiety is a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

20. The chimeric polypeptide of claim 19, wherein the tumor-associated antigen is CD19, CEA, HER2, MUC1, CD20, BCMA, ALPPL2, or EGFR.

21. The chimeric polypeptide of claim 20, wherein the tumor-associated antigen is CD19.

22. The chimeric polypeptide of any one of claims 1 to 21, wherein the ligand-inducible proteolytic cleavage site is a γ-secretase cleavage site.

23. The chimeric polypeptide of any one of claims 1 to 22, wherein the transcriptional regulator comprises a transcriptional activator, or a transcriptional repressor.

24. The chimeric polypeptide of any one of claims 1 to 23, wherein the ICD comprises a nuclear localization sequence and a transcriptional regulator sequence selected from Gal4-VP16, Gal4-VP64, tetR-VP64, ZFHD1-VP64, Gal4-KRAB, and HAP1-VP16.

25. The chimeric polypeptide of any one of claims 1 to 24, further comprising a signal sequence, a detectable label, a tumor-specific cleavage site, a disease-specific cleavage site, or a combination thereof.

26. The chimeric polypeptide of any one of claims 2 to 25, wherein the STS comprises an amino acid sequence having at least 80% sequence identity to SEQ ID NO: 85-156 and 173.

27. The chimeric polypeptide of any one of claims 2 to 25, wherein the STS comprises an amino acid sequence having at least 80% sequence identity to SEQ ID NO: 89, 92, 108, 115, 138, 146, and 156.

28. The chimeric polypeptide of any one of claims 1 to 27, wherein the hinge domain is capable of promoting oligomer formation of the chimeric polypeptide via intermolecular disulfide bonding.

29. The chimeric polypeptide of any one of claims 1 to 28, wherein the linking sequence comprises an amino acid sequence having at least 80% sequence identity to a hinge domain derived from CD8a hinge domain, a CD28 hinge domain, a CD152 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof.

30. The chimeric polypeptide of claim 29, wherein the hinge domain is derived from a CD8a hinge domain or a functional variant thereof.

31. The chimeric polypeptide of claim 30, wherein the hinge domain comprises an amino acid sequence having at least 80% sequence identity to any one of SEQ ID NOS: 157-161.

32. The chimeric polypeptide of any one of claims 1 to 31, wherein the TMD comprises an amino acid sequence having at least 80% sequence identity to any one of SEQ ID NOS: 1-84, 178, 182-185, and 190-192.

33. The chimeric polypeptide of claim 32, wherein the TMD comprises an amino acid sequence having at least 90% sequence identity to any one of SEQ ID NOS: 1-2, 6, 20, 43, 39, 54, and 64.

34. The chimeric polypeptide of any one of claims 1 to 33, wherein the TMD comprises an amino acid sequence having at least 95% sequence identity to any one of SEQ ID NOS: 1-2, 6, 20, 43, 39, 54, and 64.

35. The chimeric polypeptide of claim 34, wherein the TMD comprises an amino acid sequence substantially identical to any one of SEQ ID NOS: 1-2, 6, 20, 43, 39, 54, and 64.

36. The chimeric polypeptide of any one of claims 1 to 35, wherein:
a) the linking sequence comprises an amino acid sequence having at least 80% sequence identity to any one of SEQ ID NOS: 157-161;
b) the TMD comprises an amino acid sequence having at least 80% sequence identity to any one of SEQ ID NO: 1-84, 178, 182-185, and 190-192; and
c) the STS comprises an amino acid sequence having at least 80% sequence identity to SEQ ID NO: 85-156 and 173.

37. A recombinant nucleic acid comprising a nucleotide sequence encoding a chimeric polypeptide according to any one of claims 1 to 36.

38. The recombinant nucleic acid of claim 37, wherein the nucleotide sequence is incorporated into an expression cassette or an expression vector.

39. The recombinant nucleic acid of claim 38, wherein the expression vector is a viral vector.

40. The recombinant nucleic acid of claim 39, wherein the viral vector is a lentiviral vector, an adenovirus vector, an adeno-associated virus vector, or a retroviral vector.

41. The recombinant nucleic acid of claim 40, wherein the viral vector is a lentivival vector.

42. A recombinant cell comprising:
a) a chimeric polypeptide according to any one of claims 1 to 36; and/or
b) a recombinant nucleic acid according to any one of claims 37 to 41.

43. The recombinant cell of claim 42, wherein the cell is a mammalian cell.

44. The recombinant cell of claim 43, wherein the mammalian cell is an immune cell, a neuron, an epithelial cell, an endothelial cell, or a stem cell.

45. The recombinant cell of claim 44, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

46. The recombinant cell of any one of claims 42 to 45, further comprising:
a) a second chimeric polypeptide according to any one of claims 1 to 36; and/or
b) a second nucleic acid according to any one of claims 37 to 41;
wherein the first chimeric polypeptide and the second chimeric polypeptide do not have the same sequence, and/or the first nucleic acid or the second nucleic acid do not have the same sequence.

47. The recombinant cell of claim 46, wherein the chimeric polypeptide modulates the expression and/or activity of the second chimeric polypeptide.

48. The recombinant cell of any one of claims 42 to 47, further comprising an expression cassette encoding a protein of interest operably linked to a promoter, wherein expression of the protein is modulated by the transcriptional regulator encoded by the chimeric receptor.

49. The recombinant cell of claim 48, wherein the protein of interest is heterologous to the cell.

50. The recombinant cell of claim 48 or 49, wherein the promoter is GAL4.

51. The recombinant cell of claim 48 or 50, wherein the protein of interest is a cytokine, a cytotoxin, a chemokine, an immunomodulator, a pro-apoptotic factor, an anti-apoptotic factor, a hormone, a differentiation factor, a de-differentiation factor, an immune cell receptor, or a reporter.

52. A cell culture comprising a recombinant cell according to any one of claims 42 to 51, and a culture medium.

53. A pharmaceutical composition comprising a pharmaceutically acceptable carrier, and one or more of the following:
a) a recombinant nucleic acid according to any one of claims 37 to 41; or
b) a recombinant cell according to any one of claims 42 to 51.

54. The pharmaceutical composition of claim 53, wherein the composition comprises a recombinant nucleic acid according to any one of claims 37 to 41, and a pharmaceutically acceptable carrier.

55. The pharmaceutical composition of claim 54, wherein the recombinant nucleic acid is encapsulated in a viral capsid or a lipid nanoparticle.

56. A method for modulating an activity of a cell, the method comprising:
a) providing a recombinant cell according to any one of claims 42 to 51; and
b) contacting the recombinant cell with the selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell.

57. The method of claim 56, the contacting is carried out in vivo, ex vivo, or in vitro.

58. The method of any one of claims 56 to 57, wherein the activity of the cell is selected from the group consisting of: expression of a selected gene of the cell, proliferation of the cell, apoptosis of the cell, non-apoptotic death of the cell, differentiation of the cell, de-differentiation of the cell, migration of the cell, secretion of a molecule from the cell, cellular adhesion of the cell, and cytolytic activity of the cell.

59. The method of any one of claims 56 to 58, wherein the released transcriptional regulator modulates expression of a gene product of the cell.

60. The method of any one of claims 56 to 59, wherein the released transcriptional regulator modulates expression of a heterologous gene product.

61. The method of any one of claims 59 to 60, wherein the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin-derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

62. The method of any one of claims 56 to 61, wherein the released transcriptional regulator modulates differentiation of the cell, and wherein the cell is an immune cell, a stem cell, a progenitor cell, or a precursor cell.

63. A method for inhibiting an activity of a target cell in an individual, the method comprising administering to the individual an effective number of the recombinant cell according to any one of claims 42 to 51, wherein the recombinant cell inhibits an activity of the target cell in the individual.

64. The method of claim 63, wherein the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell.

65. The method of claim 63, wherein the target cell is a pathogenic cell.

66. A method for the treatment of a disease in an individual in need thereof, the method comprising: administering to the individual a first therapy comprising an effective number of the recombinant cell according to any one of claims 42 to 51, wherein the recombinant cell treats the disease in the individual.

67. The method of claim 66, further comprising administering to the individual a second therapy.

68. The method of claim 67, wherein the second therapy is selected from the group consisting of chemotherapy, radiotherapy, immunotherapy, hormonal therapy, or toxin therapy.

69. The method of any one of claims 66 to 68, wherein the first therapy and the second therapy are administered together, in the same composition or in separate compositions.

70. The method any one of claims 67 to 69, wherein the first therapy and the second therapy are administered simultaneously.

71. The method of any one of claims 67 to 68, wherein the first therapy and the second therapy are administered sequentially.

72. The method of claim 71, wherein the first therapy is administered before the second therapy.

73. The method of claim 71, wherein the first therapy is administered after the second therapy.

74. The method of claim 71, wherein the first therapy and the second therapy are administered in rotation.

75. A system for modulating an activity of a cell, inhibiting a target cancer cell, or treating a disease in an individual in need thereof, wherein the system comprises one or more of the following:
a) a chimeric polypeptide according to any one of claims 1 to 36;
b) a recombinant nucleic acid according to any one of claims 37 to 41;
c) a recombinant cell according to any one of claims 42 to 51; and
d) a pharmaceutical composition according to any one of claims 53 to 55.

76. A method for making the recombinant cell according to any one of claims 42 to 51, comprising:
a) providing a cell capable of protein expression; and
b) contacting the provided cell with a recombinant nucleic acid according to any one of claims 37 to 41.

77. The method of claim 76, wherein the cell is obtained by leukapheresis performed on a sample obtained from a human subject, and the cell is contacted ex vivo.

78. The method of claim 76, wherein the recombinant nucleic acid is encapsulated in a viral capsid or a lipid nanoparticle (LNP).

79. The use of one or more of the following for the treatment of a disease:
a) a chimeric polypeptide according to any one of claims 1 to 36;
b) a recombinant nucleic acid according to any one of claims 37 to 41;
c) a recombinant cell according to any one of claims 42 to 51; and
d) a composition according to any one of claims 53 to 55.

80. The use of claim 79, wherein the disease is cancer.

81. The use of the invention of any one of claims 1 to 80, for the manufacture of a medicament for the treatment of a disease.


SEQUENCE LISTING (248 sequences)

SEQ ID NO: 1 - protein, 21 aa, Artificial sequence
  Synthetic construct
  MISC_FEATURE: CLSTN1 TMD
ATVVIVVCVSFLVFMIILGVF

SEQ ID NO: 2 - protein, 22 aa, Artificial sequence
  Synthetic construct
  MISC_FEATURE: CLSTN2 TMD
IATVVIIISVCMLVFVVAMGVY

SEQ ID NO: 3 - protein, 23 aa, Artificial sequence
  Synthetic construct
  MISC_FEATURE: APLP1 TMD
AVSGLLIMGAGGGSLIVLSMLLL

SEQ ID NO: 4 - protein, 24 aa, Artificial sequence
  Synthetic construct
  MISC_FEATURE: APLP2 TMD
SALIGLLVIAVAIATVIVISLVML

SEQ ID NO: 5 - protein, 22 aa, Artificial sequence
  Synthetic construct
  MISC_FEATURE: LRP8 TMD
VIGIIVPIVVIALLCMSGYLIW

SEQ ID NO: 6 - protein, 24 aa, Artificial sequence
  Synthetic construct
  MISC_FEATURE: APP TMD
GAIIGLMVGGVVIATVIVITLVML

SEQ ID NO: 7 - protein, 24 aa, Artificial sequence
  Synthetic construct
  MISC_FEATURE: BTC TMD
ILVICLIAVMVVFIILVIGVCTCC

SEQ ID NO: 8 - protein, 23 aa, Artificial sequence
  Synthetic construct
  MISC_FEATURE: TGBR3 TMD
MGIAFAAFVIGALLTGALWYIYS

SEQ ID NO: 9 - protein, 24 aa, Artificial sequence
  Synthetic construct
  MISC_FEATURE: SPN TMD
GMLPVAVLVALLAVIVLVALLLLW

SEQ ID NO: 10 - protein, 23 aa, Artificial sequence
  Synthetic construct
  MISC_FEATURE: CD44 TMD
WLIILASLLALALILAVCIAVNS

SEQ ID NO: 11 - protein, 21 aa, Artificial sequence
  Synthetic construct
  MISC_FEATURE: CSF1R TMD
VVVACMSIMALLLLLLLLLLY

SEQ ID NO: 12 - protein, 22 aa, Artificial sequence
  Synthetic construct
  MISC_FEATURE: CXCL16 TMD
VPVLCLLAIIFILTAALSYVLC

SEQ ID NO: 13 - protein, 28 aa, Artificial sequence
  Synthetic construct
  MISC_FEATURE: CX3CL1 TMD
AVGLLAFLGLLFCLGVAMFTYQSLQGCP

SEQ ID NO: 14 - protein, 25 aa, Artificial sequence
  Synthetic construct
  MISC_FEATURE: DCC TMD
LLVIIVVTVGVITVLVVVIVAVICT

SEQ ID NO: 15 - protein, 24 aa, Artificial sequence
  Synthetic construct
  MISC_FEATURE: DLL1 TMD
VAVCAGVILVLMLLLGCAAVVVCV

SEQ ID NO: 16 - protein, 26 aa, Artificial sequence
  Synthetic construct
  MISC_FEATURE: DSG2 TMD
LGPAAIALMILAFLLLLLVPLLLLMC

SEQ ID NO: 17 - protein, 22 aa, Artificial sequence
  Synthetic construct
  MISC_FEATURE: DNER TMD
IIIGALCVAFILMLIILIVGIC

SEQ ID NO: 18 - protein, 26 aa, Artificial sequence
  Synthetic construct
  MISC_FEATURE: DAG1 TMD
YLHTVIPAVVVAAILLIAGIIAMICY

SEQ ID NO: 19 - protein, 22 aa, Artificial sequence
  Synthetic construct
  MISC_FEATURE: CDH1 TMD
ILGILGGILALLILILLLLLFL

SEQ ID NO: 20 - protein, 24 aa, Artificial sequence
  Synthetic construct
  MISC_FEATURE: EPCAM TMD
AGVIAVIVVVVIAVVAGIVVLVIS

SEQ ID NO: 21 - protein, 23 aa, Artificial sequence
  Synthetic construct
  MISC_FEATURE: EPHA4 TMD
VLLVSVSGSVVLVVILIAAFVIS

SEQ ID NO: 22 - protein, 23 aa, Artificial sequence
  Synthetic construct
  MISC_FEATURE: EPHB2 TMD
IIGSSAAGLVFLIAVVVIAIVCN

SEQ ID NO: 23 - protein, 26 aa, Artificial sequence
  Synthetic construct
  MISC_FEATURE: EFNB1 TMD
VALFAAVGAGCVIFLLIIIFLTVLLL

SEQ ID NO: 24 - protein, 21 aa, Artificial sequence
  Synthetic construct
  MISC_FEATURE: EFNB2 TMD
GIASGCIIFIVIIITLVVLLL

SEQ ID NO: 25 - protein, 24 aa, Artificial sequence
  Synthetic construct
  MISC_FEATURE: ErbB4 TMD
LIAAGVIGGLFILVIVGLTFAVYV

SEQ ID NO: 26 - protein, 24 aa, Artificial sequence
  Synthetic construct
  MISC_FEATURE: GHR TMD
FPWLLIIIFGIFGLTVMLFVFLFS

SEQ ID NO: 27 - protein, 24 aa, Artificial sequence
  Synthetic construct
  MISC_FEATURE: HLA-A TMD
VGIIAGLVLFGAVITGAVVAAVMW

SEQ ID NO: 28 - protein, 21 aa, Artificial sequence
  Synthetic construct
  MISC_FEATURE: IFNAR2 TMD
IGGIITVFLIALVLTSTIVTL

SEQ ID NO: 29 - protein, 23 aa, Artificial sequence
  Synthetic construct
  MISC_FEATURE: IGF1R TMD
LIIALPVAVLLIVGGLVIMLYVF

SEQ ID NO: 30 - protein, 20 aa, Artificial sequence
  Synthetic construct
  MISC_FEATURE: IL1R1 TMD
HMIGICVTLTVIIVCSVFIY

SEQ ID NO: 31 - protein, 26 aa, Artificial sequence
  Synthetic construct
  MISC_FEATURE: IL1R2 TMD
ASSTFSWGIVLAPLSLAFLVLGGIWM

SEQ ID NO: 32 - protein, 21 aa, Artificial sequence
  Synthetic construct
  MISC_FEATURE: IL6R TMD
TFLVAGGSLAFGTLLCIAIVL

SEQ ID NO: 33 - protein, 23 aa, Artificial sequence
  Synthetic construct
  MISC_FEATURE: INSR TMD
IIIGPLIFVFLFSVVIGSIYLFL

SEQ ID NO: 34 - protein, 25 aa, Artificial sequence
  Synthetic construct
  MISC_FEATURE: ERN1 TMD
MATIILSTFLLIGWVAFIITYPLSM

SEQ ID NO: 35 - protein, 21 aa, Artificial sequence
  Synthetic construct
  MISC_FEATURE: ERN2 TMD
QDLLAASLTAVLLGGWILFVM

SEQ ID NO: 36 - protein, 26 aa, Artificial sequence
  Synthetic construct
  MISC_FEATURE: JAG2 TMD
GLLVPVLCGAFSVLWLACVVLCVWWT

SEQ ID NO: 37 - protein, 23 aa, Artificial sequence
  Synthetic construct
  MISC_FEATURE: KCNE1 TMD
ALYVLMVLGFFGFFTLGIMLSYI

SEQ ID NO: 38 - protein, 24 aa, Artificial sequence
  Synthetic construct
  MISC_FEATURE: KCNE2 TMD
VILYLMVMIGMFSFIIVAILVSTV

SEQ ID NO: 39 - protein, 23 aa, Artificial sequence
  Synthetic construct
  MISC_FEATURE: KCNE3 TMD
YMYILFVMFLFAVTVGSLILGYT

SEQ ID NO: 40 - protein, 23 aa, Artificial sequence
  Synthetic construct
  MISC_FEATURE: KCNE4 TMD
YFYILVVMSFYGIFLIGIMLGYM

SEQ ID NO: 41 - protein, 23 aa, Artificial sequence
  Synthetic construct
  MISC_FEATURE: KL TMD
LLAFIAFLFFASIISLSLIFYYS

SEQ ID NO: 42 - protein, 21 aa, Artificial sequence
  Synthetic construct
  MISC_FEATURE: CHL1 TMD
FIGLMCAIALLTLLLLTVCFV

SEQ ID NO: 43 - protein, 21 aa, Artificial sequence
  Synthetic construct
  MISC_FEATURE: PTPRF TMD
VTGPVLAVILIILIVIAILLF

SEQ ID NO: 44 - protein, 25 aa, Artificial sequence
  Synthetic construct
  MISC_FEATURE: LRP1 TMD
HIASILIPLLLLLLLVLVAGVVFWY

SEQ ID NO: 45 - protein, 23 aa, Artificial sequence
  Synthetic construct
  MISC_FEATURE: LRP1B TMD
AIIVPLVLLVTLITTLVIGLVLC

SEQ ID NO: 46 - protein, 23 aa, Artificial sequence
  Synthetic construct
  MISC_FEATURE: LRP2 TMD
AVAVLLTILLIVVIGALAIAGFF

SEQ ID NO: 47 - protein, 25 aa, Artificial sequence
  Synthetic construct
  MISC_FEATURE: LRP6 TMD
TNTVGSVIGVIVTIFVSGTVYFICQ

SEQ ID NO: 48 - protein, 28 aa, Artificial sequence
  Synthetic construct
  MISC_FEATURE: MUC1 TMD
WGIALLVLVCVLVALAIVYLIALAVCQC

SEQ ID NO: 49 - protein, 22 aa, Artificial sequence
  Synthetic construct
  MISC_FEATURE: CDH2 TMD
IIAILLCIIILLILVLMFVVWM

SEQ ID NO: 50 - protein, 22 aa, Artificial sequence
  Synthetic construct
  MISC_FEATURE: SCN1B TMD
IMMYVLIVVLTIWLVAEMIYCY

SEQ ID NO: 51 - protein, 21 aa, Artificial sequence
  Synthetic construct
  MISC_FEATURE: SCN2B TMD
VIVGASVGGFLAVVILVLMVV

SEQ ID NO: 52 - protein, 22 aa, Artificial sequence
  Synthetic construct
  MISC_FEATURE: SCN3B TMD
IMMYILLVFLTLWLLIEMIYCY

SEQ ID NO: 53 - protein, 21 aa, Artificial sequence
  Synthetic construct
  MISC_FEATURE: SCN4B TMD
TLIILAVVGGVIGLLILILLI

SEQ ID NO: 54 - protein, 23 aa, Artificial sequence
  Synthetic construct
  MISC_FEATURE: NECTIN1 TMD
IIGGVAGSILLVLIVVGGIVVAL

SEQ ID NO: 55 - protein, 23 aa, Artificial sequence
  Synthetic construct
  MISC_FEATURE: NRG1 TMD
VLTITGICIALLVVGIMCVVAYC

SEQ ID NO: 56 - protein, 23 aa, Artificial sequence
  Synthetic construct
  MISC_FEATURE: NRG2 TMD
VLTITGICVALLVVGIVCVVAYC

SEQ ID NO: 57 - protein, 23 aa, Artificial sequence
  Synthetic construct
  MISC_FEATURE: NPR3 TMD
SAVTGIVVGALLGAGLLMAFYFF

SEQ ID NO: 58 - protein, 22 aa, Artificial sequence
  Synthetic construct
  MISC_FEATURE: Nradd TMD
IIPVYCALLATVILGLLAYVAF

SEQ ID NO: 59 - protein, 22 aa, Artificial sequence
  Synthetic construct
  MISC_FEATURE: NGFR TMD
LIPVYCSILAAVVVGLVAYIAF

SEQ ID NO: 60 - protein, 24 aa, Artificial sequence
  Synthetic construct
  MISC_FEATURE: PAM TMD
VPVVLITTLLVIPVVVLLAIAIFI

SEQ ID NO: 61 - protein, 24 aa, Artificial sequence
  Synthetic construct
  MISC_FEATURE: PLXDC2 TMD
GLIIGILILVLIVATAILVTVYMY

SEQ ID NO: 62 - protein, 24 aa, Artificial sequence
  Synthetic construct
  MISC_FEATURE: PKHD1 TMD
IILAASLSSVASWLALSCLVCCWL

SEQ ID NO: 63 - protein, 24 aa, Artificial sequence
  Synthetic construct
  MISC_FEATURE: PCDHA4 TMD
VYLIIAICAVSSLLVLTLLLYTAL

SEQ ID NO: 64 - protein, 22 aa, Artificial sequence
  Synthetic construct
  MISC_FEATURE: PCDHGC3 TMD
LLLSLILVSVGFVVTVFGVIIF

SEQ ID NO: 65 - protein, 26 aa, Artificial sequence
  Synthetic construct
  MISC_FEATURE: PTPRZ1 TMD
AVIPLVIVSALTFICLVVLVGILIYW

SEQ ID NO: 66 - protein, 22 aa, Artificial sequence
  Synthetic construct
  MISC_FEATURE: AGER TMD
LALGILGGLGTAALLIGVILWQ

SEQ ID NO: 67 - protein, 22 aa, Artificial sequence
  Synthetic construct
  MISC_FEATURE: PTPRK TMD
IAGISAGILVFILLLLVVILIV

SEQ ID NO: 68 - protein, 22 aa, Artificial sequence
  Synthetic construct
  MISC_FEATURE: PTPRM TMD
IAGVIAGILLFVIIFLGVVLVM

SEQ ID NO: 69 - protein, 22 aa, Artificial sequence
  Synthetic construct
  MISC_FEATURE: ROBO1 TMD
AFIAGIGAACWIILMVFSIWLY

SEQ ID NO: 70 - protein, 21 aa, Artificial sequence
  Synthetic construct
  MISC_FEATURE: SORCS3 TMD
AMLMLLSVVFVGLAVFLIYKF

SEQ ID NO: 71 - protein, 21 aa, Artificial sequence
  Synthetic construct
  MISC_FEATURE: SORCS1 TMD
GSAMLMLLSVVFVGLAVFVIY

SEQ ID NO: 72 - protein, 23 aa, Artificial sequence
  Synthetic construct
  MISC_FEATURE: SORL1 TMD
AVVVPILFLILLSLGVGFAILYT

SEQ ID NO: 73 - protein, 23 aa, Artificial sequence
  Synthetic construct
  MISC_FEATURE: SORT1 TMD
SVPIILAIVGLMLVTVVAGVLIV

SEQ ID NO: 74 - protein, 22 aa, Artificial sequence
  Synthetic construct
  MISC_FEATURE: SDC1 TMD
GVIAGGLVGLIFAVCLVGFMLY

SEQ ID NO: 75 - protein, 25 aa, Artificial sequence
  Synthetic construct
  MISC_FEATURE: SDC2 TMD
VLAAVIAGGVIGFLFAIFLILLLVY

SEQ ID NO: 76 - protein, 22 aa, Artificial sequence
  Synthetic construct
  MISC_FEATURE: SDC3 TMD
AVIVGGVVGALFAAFLVTLLIY

SEQ ID NO: 77 - protein, 27 aa, Artificial sequence
  Synthetic construct
  MISC_FEATURE: TIE1 TMD
QLILAVVGSVSATCLTILAALLTLVCI

SEQ ID NO: 78 - protein, 24 aa, Artificial sequence
  Synthetic construct
  MISC_FEATURE: TYR TMD
WLLGAAMVGAVLTALLAGLVSLLC

SEQ ID NO: 79 - protein, 24 aa, Artificial sequence
  Synthetic construct
  MISC_FEATURE: TYRP1 TMD
IIAIAVVGALLLVALIFGTASYLI

SEQ ID NO: 80 - protein, 23 aa, Artificial sequence
  Synthetic construct
  MISC_FEATURE: DCT TMD
LLVVMGTLVALVGLFVLLAFLQY

SEQ ID NO: 81 - protein, 24 aa, Artificial sequence
  Synthetic construct
  MISC_FEATURE: VASN TMD
LLIAPALAAVLLAALAAVGAAYCV

SEQ ID NO: 82 - protein, 21 aa, Artificial sequence
  Synthetic construct
  MISC_FEATURE: CDH5 TMD
AVVAILLCILTITVITLLIFL

SEQ ID NO: 83 - protein, 22 aa, Artificial sequence
  Synthetic construct
  MISC_FEATURE: FLT1 TMD
LITLTCTCVAATLFWLLLTLFI

SEQ ID NO: 84 - protein, 22 aa, Artificial sequence
  Synthetic construct
  MISC_FEATURE: VLDLR TMD
AAWAILPLLLLVMAAVGGYLMW

SEQ ID NO: 85 - protein, 4 aa, Artificial sequence
  Synthetic construct
  MISC_FEATURE: APLP1 STS
RRKK

SEQ ID NO: 86 - protein, 3 aa, Artificial sequence
  Synthetic construct
  MISC_FEATURE: APLP2 STS
RKR

SEQ ID NO: 87 - protein, 3 aa, Artificial sequence
  Synthetic construct
  MISC_FEATURE: APP STS
KKK

SEQ ID NO: 88 - protein, 1 aa, Artificial sequence
  Synthetic construct
  MISC_FEATURE: TGBR3 STS
H

SEQ ID NO: 89 - protein, 7 aa, Artificial sequence
  Synthetic construct
  MISC_FEATURE: CSF1R STS
KYKQKPK

SEQ ID NO: 90 - protein, 4 aa, Artificial sequence
  Synthetic construct
  MISC_FEATURE: CXCL16 STS
KRRR

SEQ ID NO: 91 - protein, 2 aa, Artificial sequence
  Synthetic construct
  MISC_FEATURE: CX3CL1 STS
RK

SEQ ID NO: 92 - protein, 7 aa, Artificial sequence
  Synthetic construct
  MISC_FEATURE: DAG1 STS
RKKRKGK

SEQ ID NO: 93 - protein, 2 aa, Artificial sequence
  Synthetic construct
  MISC_FEATURE: DCC STS
RR

SEQ ID NO: 94 - protein, 4 aa, Artificial sequence
  Synthetic construct
  MISC_FEATURE: DNER STS
RISR

SEQ ID NO: 95 - protein, 7 aa, Artificial sequence
  Synthetic construct
  MISC_FEATURE: DSG2 STS
KCGKGAK

SEQ ID NO: 96 - protein, 3 aa, Artificial sequence
  Synthetic construct
  MISC_FEATURE: CDH1 STS
RRR

SEQ ID NO: 97 - protein, 6 aa, Artificial sequence
  Synthetic construct
  MISC_FEATURE: GHR STS
KQQRIK

SEQ ID NO: 98 - protein, 3 aa, Artificial sequence
  Synthetic construct
  MISC_FEATURE: HLA-A STS
RRK

SEQ ID NO: 99 - protein, 1 aa, Artificial sequence
  Synthetic construct
  MISC_FEATURE: IFNAR2 STS
K

SEQ ID NO: 100 - protein, 4 aa, Artificial sequence
  Synthetic construct
  MISC_FEATURE: IGF1R STS
HRKR

SEQ ID NO: 101 - protein, 4 aa, Artificial sequence
  Synthetic construct
  MISC_FEATURE: IL1R1 STS
KIFK

SEQ ID NO: 102 - protein, 1 aa, Artificial sequence
  Synthetic construct
  MISC_FEATURE: ERN2 STS
R

SEQ ID NO: 103 - protein, 7 aa, Artificial sequence
  Synthetic construct
  MISC_FEATURE: KCNE1 STS
RSKKLEH

SEQ ID NO: 104 - protein, 7 aa, Artificial sequence
  Synthetic construct
  MISC_FEATURE: KCNE2 STS
KSKRREH

SEQ ID NO: 105 - protein, 7 aa, Artificial sequence
  Synthetic construct
  MISC_FEATURE: CHL1 STS
KRNRGGK

SEQ ID NO: 106 - protein, 3 aa, Artificial sequence
  Synthetic construct
  MISC_FEATURE: LRP1 STS
KRR

SEQ ID NO: 107 - protein, 4 aa, Artificial sequence
  Synthetic construct
  MISC_FEATURE: LRP2 STS
HYRR

SEQ ID NO: 108 - protein, 6 aa, Artificial sequence
  Synthetic construct
  MISC_FEATURE: PTPRF STS
KRKRTH

SEQ ID NO: 109 - protein, 2 aa, Artificial sequence
  Synthetic construct
  MISC_FEATURE: SCN1B STS
KK

SEQ ID NO: 110 - protein, 5 aa, Artificial sequence
  Synthetic construct
  MISC_FEATURE: SCN3B STS
RKVSK

SEQ ID NO: 111 - protein, 5 aa, Artificial sequence
  Synthetic construct
  MISC_FEATURE: NPR3 STS
RKKYR

SEQ ID NO: 112 - protein, 2 aa, Artificial sequence
  Synthetic construct
  MISC_FEATURE: NGFR STS
KR

SEQ ID NO: 113 - protein, 2 aa, Artificial sequence
  Synthetic construct
  MISC_FEATURE: PLXDC2 STS
HH

SEQ ID NO: 114 - protein, 6 aa, Artificial sequence
  Synthetic construct
  MISC_FEATURE: PAM STS
RWKKSR

SEQ ID NO: 115 - protein, 5 aa, Artificial sequence
  Synthetic construct
  MISC_FEATURE: AGER STS
RRQRR

SEQ ID NO: 116 - protein, 6 aa, Artificial sequence
  Synthetic construct
  MISC_FEATURE: ROBO1 STS
RHRKKR

SEQ ID NO: 117 - protein, 3 aa, Artificial sequence
  Synthetic construct
  MISC_FEATURE: SORCS3 STS
KRK

SEQ ID NO: 118 - protein, 5 aa, Artificial sequence
  Synthetic construct
  MISC_FEATURE: SORCS1 STS
KFKRR

SEQ ID NO: 119 - protein, 4 aa, Artificial sequence
  Synthetic construct
  MISC_FEATURE: SORL1 STS
KHRR

SEQ ID NO: 120 - protein, 5 aa, Artificial sequence
  Synthetic construct
  MISC_FEATURE: SDC1 STS
RMKKK

SEQ ID NO: 121 - protein, 5 aa, Artificial sequence
  Synthetic construct
  MISC_FEATURE: SDC2 STS
RMRKK

SEQ ID NO: 122 - protein, 7 aa, Artificial sequence
  Synthetic construct
  MISC_FEATURE: SPN STS
RRRQKRR

SEQ ID NO: 123 - protein, 5 aa, Artificial sequence
  Synthetic construct
  MISC_FEATURE: TYR STS
RHKRK

SEQ ID NO: 124 - protein, 4 aa, Artificial sequence
  Synthetic construct
  MISC_FEATURE: TYRP1 STS
RARR

SEQ ID NO: 125 - protein, 5 aa, Artificial sequence
  Synthetic construct
  MISC_FEATURE: DCT STS
RRLRK

SEQ ID NO: 126 - protein, 4 aa, Artificial sequence
  Synthetic construct
  MISC_FEATURE: VASN STS
RRGR

SEQ ID NO: 127 - protein, 5 aa, Artificial sequence
  Synthetic construct
  MISC_FEATURE: FLT1 STS
RKMKR

SEQ ID NO: 128 - protein, 13 aa, Artificial sequence
  Synthetic construct
  MISC_FEATURE: CDH5 STS
RRRLRKQARAHGK

SEQ ID NO: 129 - protein, 9 aa, Artificial sequence
  Synthetic construct
  MISC_FEATURE: PKHD1 STS
KRSKSRKTK

SEQ ID NO: 130 - protein, 8 aa, Artificial sequence
  Synthetic construct
  MISC_FEATURE: NECTIN1 STS
RRRRHTFK

SEQ ID NO: 131 - protein, 8 aa, Artificial sequence
  Synthetic construct
  MISC_FEATURE: KL STS
KKGRRSYK

SEQ ID NO: 132 - protein, 15 aa, Artificial sequence
  Synthetic construct
  MISC_FEATURE: IL6R STS
RFKKTWKLRALKEGK

SEQ ID NO: 133 - protein, 9 aa, Artificial sequence
  Synthetic construct
  MISC_FEATURE: EFNB1 STS
KLRKRHRKH

SEQ ID NO: 134 - protein, 9 aa, Artificial sequence
  Synthetic construct
  MISC_FEATURE: CD44 STS
RRRCGQKKK

SEQ ID NO: 135 - protein, 11 aa, Artificial sequence
  Synthetic construct
  MISC_FEATURE: CLSTN1 STS
RIRAAHRRTMR

SEQ ID NO: 136 - protein, 9 aa, Artificial sequence
  Synthetic construct
  MISC_FEATURE: LRP8 STS
RNWKRKNTK

SEQ ID NO: 137 - protein, 9 aa, Artificial sequence
  Synthetic construct
  MISC_FEATURE: PCDHGC3 STS
KVYKWKQSR

SEQ ID NO: 138 - protein, 14 aa, Artificial sequence
  Synthetic construct
  MISC_FEATURE: NRG1 STS
KTKKQRKKLHDRLR

SEQ ID NO: 139 - protein, 11 aa, Artificial sequence
  Synthetic construct
  MISC_FEATURE: LRP1B STS
KRKRRTKTIRR

SEQ ID NO: 140 - protein, 14 aa, Artificial sequence
  Synthetic construct
  MISC_FEATURE: JAG2 STS
RKRRKERERSRLPR

SEQ ID NO: 141 - protein, 9 aa, Artificial sequence
  Synthetic construct
  MISC_FEATURE: EFNB2 STS
KYRRRHRKH

SEQ ID NO: 142 - protein, 8 aa, Artificial sequence
  Synthetic construct
  MISC_FEATURE: DLL1 STS
RLRLQKHR

SEQ ID NO: 143 - protein, 8 aa, Artificial sequence
  Synthetic construct
  MISC_FEATURE: CLSTN2 STS
RVRIAHQH

SEQ ID NO: 144 - protein, 10 aa, Artificial sequence
  Synthetic construct
  MISC_FEATURE: EPCAM STS
RKKRMAKYEK

SEQ ID NO: 145 - protein, 13 aa, Artificial sequence
  Synthetic construct
  MISC_FEATURE: ErbB4 STS
RRKSIKKKRALRR

SEQ ID NO: 146 - protein, 8 aa, Artificial sequence
  Synthetic construct
  MISC_FEATURE: KCNE3 STS
RSRKVDKR

SEQ ID NO: 147 - protein, 10 aa, Artificial sequence
  Synthetic construct
  MISC_FEATURE: CDH2 STS
KRRDKERQAK

SEQ ID NO: 148 - protein, 14 aa, Artificial sequence
  Synthetic construct
  MISC_FEATURE: NRG2 STS
KTKKQRKQMHNHLR

SEQ ID NO: 149 - protein, 10 aa, Artificial sequence
  Synthetic construct
  MISC_FEATURE: PTPRK STS
KKSKLAKKRK

SEQ ID NO: 150 - protein, 12 aa, Artificial sequence
  Synthetic construct
  MISC_FEATURE: BTC STS
HPLRKRRKRKKK

SEQ ID NO: 151 - protein, 10 aa, Artificial sequence
  Synthetic construct
  MISC_FEATURE: EPHA4 STS
RRRSKYSKAK

SEQ ID NO: 152 - protein, 10 aa, Artificial sequence
  Synthetic construct
  MISC_FEATURE: IL1R2 STS
HRRCKHRTGK

SEQ ID NO: 153 - protein, 8 aa, Artificial sequence
  Synthetic construct
  MISC_FEATURE: KCNE4 STS
KSKRREKK

SEQ ID NO: 154 - protein, 10 aa, Artificial sequence
  Synthetic construct
  MISC_FEATURE: SCN2B STS
KCVRRKKEQK

SEQ ID NO: 155 - protein, 9 aa, Artificial sequence
  Synthetic construct
  MISC_FEATURE: Nradd STS
KCWRSHKQR

SEQ ID NO: 156 - protein, 10 aa, Artificial sequence
  Synthetic construct
  MISC_FEATURE: PTPRM STS
KKRKLAKKRK

SEQ ID NO: 157 - protein, 45 aa, Artificial sequence
  Synthetic construct
  MISC_FEATURE: CD8a hinge
TTTPAPRPPTPAPTIASQPLSLRPEACRPAAGGAVHTRGLDFACD

SEQ ID NO: 158 - protein, 27 aa, Artificial sequence
  Synthetic construct
  MISC_FEATURE: truncated CD8a hinge
TTTPAPRPPTPAPTIASQPLSLRPEAC

SEQ ID NO: 159 - protein, 39 aa, Artificial sequence
  Synthetic construct
  MISC_FEATURE: hinge domain
IEVMYPPPYLDNEKSNGTIIHVKGKHLCPSPLFPGPSKP

SEQ ID NO: 160 - protein, 27 aa, Artificial sequence
  Synthetic construct
  MISC_FEATURE: hinge domain
GGGGSGGGGSGGGGSESKYGPPCPPCP

SEQ ID NO: 161 - protein, 186 aa, Artificial sequence [sequence omitted]

SEQ ID NO: 162 - protein, 21 aa, Artificial sequence
  Synthetic construct
  MISC_FEATURE: CD8alpha signal peptide
MALPVTALLLPLALLLHAARP

SEQ ID NO: 163 - protein, 10 aa, Artificial sequence
  Synthetic construct
  MISC_FEATURE: myc-tag
EQKLISEEDL

SEQ ID NO: 164 - protein, 20 aa, Artificial sequence
  Synthetic construct
  MISC_FEATURE: GAL4 DBD recognition motif
GGAGCACTGTCCTCCGAACG

SEQ ID NO: 165 - protein, 4 aa, Artificial sequence
  Synthetic construct
  MISC_FEATURE: Exemplary cleavage sequence of MMP-9
  MISC_FEATURE (2)..(3): Xaa can be any naturally occurring amino acid
  MISC_FEATURE (4)..(4): Xaa is Leu, Ile, Val, Phe, Trp, Tyr, Val, Met, or Pro
PXXX

SEQ ID NO: 166 - protein, 5 aa, Artificial sequence
  Synthetic construct
  MISC_FEATURE: Exemplary cleavage sequence of MMP-9
  MISC_FEATURE (2)..(4): Xaa can be any naturally occurring amino acid
  MISC_FEATURE (5)..(5): Xaa is Ser or Thr
PXXXX

SEQ ID NO: 167 - protein, 6 aa, Artificial sequence
  Synthetic construct
  MISC_FEATURE: Exemplary cleavage sequence of MMP-9
  MISC_FEATURE (2)..(2): Xaa is Leu or Gln
PXGMTS

SEQ ID NO: 168 - protein, 5 aa, Artificial sequence
  Synthetic construct
  MISC_FEATURE: Exemplary cleavage sequence of MMP-9
  MISC_FEATURE (2)..(2): Xaa is Leu or Gln
PXGMT

SEQ ID NO: 169 - protein, 3 aa, Artificial sequence
  Synthetic construct
  MISC_FEATURE: exemplary urokinase-type plasminogen activator (uPA) or tissue plasminogen activator (tPA) cleavage site
VGR

SEQ ID NO: 170 - protein, 7 aa, Artificial sequence
  Synthetic construct
  MISC_FEATURE: exemplary tobacco etch virus (TEV) protease cleavage site
ENLYTQS

SEQ ID NO: 171 - protein, 5 aa, Artificial sequence
  Synthetic construct
  MISC_FEATURE: exemplary enterokinase cleavage site
DDDDK

SEQ ID NO: 172 - protein, 4 aa, Artificial sequence
  Synthetic construct
  MISC_FEATURE: exemplary thrombin cleavage site
LVPR

SEQ ID NO: 173 - protein, 22 aa, Artificial sequence
  Synthetic construct
  MISC_FEATURE: Wild-type Notch1 TMD
FMYVAAAAFVLLFFVGCGVLLS

SEQ ID NO: 174 - protein, 273 aa, Artificial sequence
  Synthetic construct
  MISC_FEATURE: ECD
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 175 - protein, 12 aa, Artificial sequence
  Synthetic construct
  MISC_FEATURE: IgG4 N-JMD
ESKYGPPCPPCP

SEQ ID NO: 176 - protein, 24 aa, Artificial sequence
  Synthetic construct
  MISC_FEATURE: mutated Notch1_GIVVLLSmut TMD (a site from EPCAM/NECTIN1 that maintains cleavage (GIVV) was added)
FMYVAAAAFVLLFFVGCGIVVLLS

SEQ ID NO: 177 - protein, 22 aa, Artificial sequence
  Synthetic construct
  MISC_FEATURE: mutated Notch1_GIVVSmut TMD (a site from EPCAM/NECTIN1 that maintains cleavage (GIVV) was added)
FMYVAAAAFVLLFFVGCGIVVS

SEQ ID NO: 178 - protein, 24 aa, Artificial sequence
  Synthetic construct
  MISC_FEATURE: APP TMD
GAIIGLMVGGVVIATVIVITGVML

SEQ ID NO: 179 - protein, 23 aa, Artificial sequence
  Synthetic construct
  MISC_FEATURE: Notch2 TMD
LLYLLAVAVVIILFIILLGVIMA

SEQ ID NO: 180 - protein, 22 aa, Artificial sequence
  Synthetic construct
  MISC_FEATURE: Notch3 TMD
LPLLVAGAVLLLVILVLGVMVA

SEQ ID NO: 181 - protein, 24 aa, Artificial sequence
  Synthetic construct
  MISC_FEATURE: Notch3 TMD
PVLCSPVAGVILLALGALLVLQLI

SEQ ID NO: 182 - protein, 21 aa, Artificial sequence
  Synthetic construct
  MISC_FEATURE: mutated AGER _GVLVSmut TMD (the last 4 amino acids of the wild-type AGER TMD were replaced with “LVS” to functionalize the TMD)
LALGILGGLGTAALLIGVLVS

SEQ ID NO: 183 - protein, 22 aa, Artificial sequence
  Synthetic construct
  MISC_FEATURE: mutated AGER _LLmut TMD (the last 4 amino acids of the wild-type AGER TMD were replaced with “LVS” to functionalize the TMD)
LALGILGGLLLAALLIGVILWQ

SEQ ID NO: 184 - protein, 23 aa, Artificial sequence
  Synthetic construct
  MISC_FEATURE: mutated ROBO1 TMD (the last 4 amino acids of the wild-type Robo1 TMD were replaced with “GVLLS” to functionalize the TMD
AFIAGIGAACWIILMVFSGVLLS

SEQ ID NO: 185 - protein, 24 aa, Artificial sequence
  Synthetic construct
  MISC_FEATURE: Notch1 D. rerio TMD
MYPMFLVLLALAVLALAAVGVVVS

SEQ ID NO: 186 - protein, 22 aa, Artificial sequence
  Synthetic construct
  MISC_FEATURE: Notch1 D. melanogaster TMD
VITGIILVIIALAFFGMVLSTQ

SEQ ID NO: 187 - protein, 22 aa, Artificial sequence
  Synthetic construct
  MISC_FEATURE: Notch1 X. laevis TMD
PMLSMLVIPLLIIFVFMMVIVN

SEQ ID NO: 188 - protein, 22 aa, Artificial sequence
  Synthetic construct
  MISC_FEATURE: Notch1 G. gallus TMD
PMYVVVAALVLLAFIGVGVLVS

SEQ ID NO: 189 - protein, 22 aa, Artificial sequence
  Synthetic construct
  MISC_FEATURE: Notch1 M. muculs TMD
LMYVAAAAFVLLFFVGCGVLLS

SEQ ID NO: 190 - protein, 21 aa, Artificial sequence
  Synthetic construct
  MISC_FEATURE: CD8a TMD
IYIWAPLAGTCGVLLLSLVIT

SEQ ID NO: 191 - protein, 26 aa, Artificial sequence
  Synthetic construct
  MISC_FEATURE: mutated CD8a TMD
IYIWAPLAGTCGVLLLSLVITGVLLS

SEQ ID NO: 192 - protein, 23 aa, Artificial sequence
  Synthetic construct
  MISC_FEATURE: mutated CD8a TMD
IYIWAPLAGTCGVLLLSLGVLVS

SEQ ID NO: 193 - protein, 5 aa, Artificial sequence
  Synthetic construct
  MISC_FEATURE: Notch1 STS
RKRRR

SEQ ID NO: 194 - protein, 6 aa, Artificial sequence
  Synthetic construct
  MISC_FEATURE: Notch3 STS
RRKREH

SEQ ID NO: 195 - protein, 6 aa, Artificial sequence
  Synthetic construct
  MISC_FEATURE: Notch4 STS
RRRREH

SEQ ID NO: 196 - protein, 211 aa, Artificial sequence
  Synthetic construct
  MISC_FEATURE: Gal4-VP64
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 197 - protein, 558 aa, Artificial sequence
  Synthetic construct
  MISC_FEATURE: pIZ343GIVV
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFMYVAAAAFVLLFFVGCGIVVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 198 - protein, 556 aa, Artificial sequence
  Synthetic construct
  MISC_FEATURE: pIZ343GIVVa
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFMYVAAAAFVLLFFVGCGIVVSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 199 - protein, 523 aa, Artificial sequence
  Synthetic construct
  MISC_FEATURE: pIZ373
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 200 - protein, 539 aa, Artificial sequence
  Synthetic construct
  MISC_FEATURE: pIZ377
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIATVVIIISVCMLVFVVAMGVYKRKRTHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 201 - protein, 538 aa, Artificial sequence
  Synthetic construct
  MISC_FEATURE: pIZ378
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIATVVIIISVCMLVFVVAMGVYRRQRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 202 - protein, 541 aa, Artificial sequence
  Synthetic construct
  MISC_FEATURE: pIZ379
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIATVVIIISVCMLVFVVAMGVYRSRKVDKRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 203 - protein, 540 aa, Artificial sequence
  Synthetic construct
  MISC_FEATURE: pTMD206
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACGAIIGLMVGGVVIATVIVITLVMLRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 204 - protein, 540 aa, Artificial sequence
  Synthetic construct
  MISC_FEATURE: pTMD206GV
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACGAIIGLMVGGVVIATVIVITGVMLRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 205 - protein, 540 aa, Artificial sequence
  Synthetic construct
  MISC_FEATURE: pTMD207
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACILVICLIAVMVVFIILVIGVCTCCRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 206 - protein, 544 aa, Artificial sequence
  Synthetic construct
  MISC_FEATURE: pTMD213
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAVGLLAFLGLLFCLGVAMFTYQSLQGCPRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 207 - protein, 541 aa, Artificial sequence
  Synthetic construct
  MISC_FEATURE: pTMD214
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLLVIIVVTVGVITVLVVVIVAVICTRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 208 - protein, 540 aa, Artificial sequence
  Synthetic construct
  MISC_FEATURE: pTMD215
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVAVCAGVILVLMLLLGCAAVVVCVRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 209 - protein, 540 aa, Artificial sequence
  Synthetic construct
  MISC_FEATURE: pTMD220
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAGVIAVIVVVVIAVVAGIVVLVISRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 210 - protein, 540 aa, Artificial sequence
  Synthetic construct
  MISC_FEATURE: pTMD225
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLIAAGVIGGLFILVIVGLTFAVYVRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 211 - protein, 541 aa, Artificial sequence
  Synthetic construct
  MISC_FEATURE: pTMD244
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACHIASILIPLLLLLLLVLVAGVVFWYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 212 - protein, 541 aa, Artificial sequence
  Synthetic construct
  MISC_FEATURE: pTMD247
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACTNTVGSVIGVIVTIFVSGTVYFICQRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 213 - protein, 537 aa, Artificial sequence
  Synthetic construct
  MISC_FEATURE: pTMD253
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACTLIILAVVGGVIGLLILILLIRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 214 - protein, 539 aa, Artificial sequence
  Synthetic construct
  MISC_FEATURE: pTMD254
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIIGGVAGSILLVLIVVGGIVVALRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 215 - protein, 539 aa, Artificial sequence
  Synthetic construct
  MISC_FEATURE: pTMD258
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLLYLLAVAVVIILFIILLGVIMARKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 216 - protein, 538 aa, Artificial sequence
  Synthetic construct
  MISC_FEATURE: pTMD259
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLPLLVAGAVLLLVILVLGVMVARKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 217 - protein, 540 aa, Artificial sequence
  Synthetic construct
  MISC_FEATURE: pTMD260
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACPVLCSPVAGVILLALGALLVLQLIRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 218 - protein, 540 aa, Artificial sequence
  Synthetic construct
  MISC_FEATURE: pTMD260GV
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACPVLCSPVAGVILLALGALGVLQLIRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 219 - protein, 538 aa, Artificial sequence
  Synthetic construct
  MISC_FEATURE: pTMD268
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLLLSLILVSVGFVVTVFGVIIFRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 220 - protein, 538 aa, Artificial sequence
  Synthetic construct
  MISC_FEATURE: pTMD270
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLALGILGGLGTAALLIGVILWQRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 221 - protein, 537 aa, Artificial sequence
  Synthetic construct
  MISC_FEATURE: pTMD270GVLVS
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLALGILGGLGTAALLIGVLVSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 222 - protein, 538 aa, Artificial sequence
  Synthetic construct
  MISC_FEATURE: pTMD270LL
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLALGILGGLLLAALLIGVILWQRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 223 - protein, 538 aa, Artificial sequence
  Synthetic construct
  MISC_FEATURE: pTMD272
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIAGVIAGILLFVIIFLGVVLVMRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 224 - protein, 538 aa, Artificial sequence
  Synthetic construct
  MISC_FEATURE: pTMD273
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAFIAGIGAACWIILMVFSIWLYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 225 - protein, 538 aa, Artificial sequence
  Synthetic construct
  MISC_FEATURE: pTMD273GV
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAFIAGIGAACWIILMVFSGVLYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 226 - protein, 539 aa, Artificial sequence
  Synthetic construct
  MISC_FEATURE: pTMD273GVLLS
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAFIAGIGAACWIILMVFSGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 227 - protein, 539 aa, Artificial sequence
  Synthetic construct
  MISC_FEATURE: pTMD276
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAVVVPILFLILLSLGVGFAILYTRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 228 - protein, 539 aa, Artificial sequence
  Synthetic construct
  MISC_FEATURE: pTMD277
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACSVPIILAIVGLMLVTVVAGVLIVRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 229 - protein, 538 aa, Artificial sequence
  Synthetic construct
  MISC_FEATURE: pTMD278
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACGVIAGGLVGLIFAVCLVGFMLYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 230 - protein, 541 aa, Artificial sequence
  Synthetic construct
  MISC_FEATURE: pTMD279
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVLAAVIAGGVIGFLFAIFLILLLVYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 231 - protein, 538 aa, Artificial sequence
  Synthetic construct
  MISC_FEATURE: pTMD280
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACAVIVGGVVGALFAAFLVTLLIYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 232 - protein, 540 aa, Artificial sequence
  Synthetic construct
  MISC_FEATURE: pTMD289
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACMYPMFLVLLALAVLALAAVGVVVSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 233 - protein, 538 aa, Artificial sequence
  Synthetic construct
  MISC_FEATURE: pTMD290
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVITGIILVIIALAFFGMVLSTQRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 234 - protein, 538 aa, Artificial sequence
  Synthetic construct
  MISC_FEATURE: pTMD291
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACPMLSMLVIPLLIIFVFMMVIVNRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 235 - protein, 538 aa, Artificial sequence
  Synthetic construct
  MISC_FEATURE: pTMD292
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACPMYVVVAALVLLAFIGVGVLVSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 236 - protein, 538 aa, Artificial sequence
  Synthetic construct
  MISC_FEATURE: pTMD295
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 237 - protein, 555 aa, Artificial sequence
  Synthetic construct
  MISC_FEATURE: pTMD297pre
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 238 - protein, 560 aa, Artificial sequence
  Synthetic construct
  MISC_FEATURE: pTMD297pre_GV
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 239 - protein, 557 aa, Artificial sequence
  Synthetic construct
  MISC_FEATURE: pTMD297pre_GVLVS
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLGVLVSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 240 - protein, 540 aa, Artificial sequence
  Synthetic construct
  MISC_FEATURE: pSTS205
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKYKQKPKMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 241 - protein, 538 aa, Artificial sequence
  Synthetic construct
  MISC_FEATURE: pSTS231
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRRQRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 242 - protein, 541 aa, Artificial sequence
  Synthetic construct
  MISC_FEATURE: pSTS262
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRSRKVDKRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 243 - protein, 543 aa, Artificial sequence
  Synthetic construct
  MISC_FEATURE: pSTS263
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRRDKERQAKMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 244 - protein, 545 aa, Artificial sequence
  Synthetic construct
  MISC_FEATURE: pSTS266
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSHPLRKRRKRKKKMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 245 - protein, 543 aa, Artificial sequence
  Synthetic construct
  MISC_FEATURE: pSTS267
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRRRSKYSKAKMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 246 - protein, 537 aa, Artificial sequence
  Synthetic construct
  MISC_FEATURE: pSTS268
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKIFKMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 247 - protein, 539 aa, Artificial sequence
  Synthetic construct
  MISC_FEATURE: pSTS274
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRRKREHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 248 - protein, 539 aa, Artificial sequence
  Synthetic construct
  MISC_FEATURE: pIZ364
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRRRREHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS